(19) United States
(12) Patent Application Publication
(10) Pub. No.: US 2025/0345061 A1
(43) Pub. Date: Nov. 13, 2025

(54) NEURORRHAPHY SYSTEMS, DEVICES, AND METHODS

(71) Applicant: RENERVA, LLC, Pittsburgh, PA (US)

(72) Inventors: Lorenzo Soletti, Pittsburgh, PA (US); Victoria Turchick, Pittsburgh, PA (US); Nicole Cwalina, Pittsburgh, PA (US); Brandon Burger, Pittsburgh, PA (US); Mark Mahan, Salt Lake City, UT (US); Howard Schrayer, Hilton Head Island, NC (US); John Wainwright, Foothill, CA (US)

(21) Appl. No.: 18/722,959

(22) PCT Filed: Dec. 30, 2022
(86) PCT No.: PCT/US2022/054351

§ 371 Date: Jun. 21, 2024

Related U.S. Application Data
(60) Provisional application No. 63/295,642, filed on Dec. 31, 2021.

(51) Int. Cl. A61B 17/11; A61B 17/00

(52) U.S. Cl. CPC A61B 17/1128; A61B 2017/00004; A61B 2017/1103; A61B 2017/1132

(57) ABSTRACT

Systems, devices, and methods for connecting two nerve stumps are provided. A system includes at least one neurorrhaphy device constructed and arranged to engage at least a portion of a proximal nerve stump and a distal nerve stump. The neurorrhaphy device is deployed at one, two, or more deposit sites within a patient, and provides a therapeutic benefit at the deposit site. The neurorrhaphy device allows for the alignment and/or reapproximation the proximal and distal nerve stumps without the application of sutures or other tissue penetrating components within, or in immediate proximity to, the nerve stumps.

### RELATED APPLICATIONS

This application claims benefit of U.S. Provisional patent application Ser. No. 63/295,642, entitled “Neurorrhaphy Systems, Devices, and Methods”, filed Dec. 31, 2021, the content of each of which is incorporated herein by reference in its entirety for all purposes.

### This Application is Related to:

U.S. Pat. No. 8,361,503, Issued Jan. 29, 2013;
U.S. Pat. No. 8,691,276, Issued Apr. 8, 2014;
U.S. Pat. No. 9,737,635, Issued Aug. 22, 2017;
U.S. Pat. No. 10,004,827, Issued Jun. 26, 2018;
U.S. Pat. No. 10,179,192, Issued Jan. 15, 2019;
U.S. Pat. No. 10,213,526, Issued Feb. 26, 2019;
U.S. Pat. No. 10,729,813, Issued Aug. 4, 2020; and
U.S. Pat. No. 10,772,989, Issued Sep. 15, 2020;
the content of each of which is incorporated herein by reference in its entirety for all purposes.

### This Application is Related to:

U.S. patent application Ser. No. 15/996,916, entitled “Extracellular Matrix-Derived Gels and Related Methods”, filed Jun. 4, 2018, published as US2019/0038803, published Feb. 7, 2019;
U.S. patent application Ser. No. 16/288,831, entitled “Extracellular Matrix-Derived Gels and Related Methods”, filed Feb. 28, 2019, published as US2019/0201581, published Jul. 4, 2019;
U.S. patent application Ser. No. 16/992,442, entitled “Injectable Peripheral Nerve Specific Hydrogel”, filed Aug. 13, 2020, published as US2021/0196860, published Jul. 1, 2021;
U.S. patent application Ser. No. 16/238,826, entitled “Methods for Preparation of a Terminally Sterilized Hydrogel Derived from Extracellular Matrix”, filed Jan. 3, 2019, published as US2019/0374683, published Dec. 12, 2019;
International PCT patent application Serial Number PCT/US2020/067431, entitled “Extracellular Matrix Systems, Devices, and Methods of Deployment”, filed Dec. 30, 2020, published as WO2021/0138399, published Jul. 8, 2021;
U.S. patent application Ser. No. 17/788,450, entitled “Extracellular Matrix Systems, Devices, and Methods of Deployment” filed Jun. 23, 2022, published as US published
International PCT patent application Serial Number PCT/US2020/053570, entitled “Extracellular Matrix Devices and Methods of Manufacture”, filed Sep. 30, 2020, published as WO2021/067456, published Apr. 8, 2021;
U.S. patent application Ser. No. 17/762,858, entitled “Extracellular Matrix Devices and Methods of Manufacture” filed Mar. 23, 2022, published as US 2022/0323648, published Oct. 13, 2022;
U.S. Provisional patent application Ser. No. 63/250,379, entitled “Nerve Graft Systems, Devices, and Methods”, filed Sep. 30, 2021;
U.S. Provisional patent application Ser. No. 63/329,597, entitled “Nerve Graft Systems, Devices, and Methods”, filed Apr. 11, 2022; and
International PCT patent application Serial Number PCT/US2022/045365, entitled “Nerve Graft Systems, Devices, and Methods”, filed Sep. 30, 2022, published as WO published the content of each of which is incorporated by reference in its entirety for all purposes.

### FIELD OF INVENTIVE CONCEPTS

The present inventive concepts relate generally to systems, devices, and methods for the alignment and reapproximation of peripheral nerve segments without the need for applying sutures or other materials within, or in immediate proximity to, the area where the nerve transection has occurred.

### BACKGROUND

Acute peripheral nerve injuries frequently result in permanent disability. Such injuries arise from blunt, compression and/or stretch traumas (e.g. motor vehicle crashes), deep tissue lacerations (e.g. power tool or sharp tool accidents), focal injuries (e.g. gun shots, penetrating body injuries), or iatrogenic injuries (e.g. medically-induced) resulting from tumor resections or other surgical procedures or treatments. These injuries are associated with loss of motor and/or sensory function, chronic pain, and combinations thereof, and yield a progressively worse prognosis with increasing severity of the injury.

Surgeons performing peripheral nerve repair procedures to treat these acute injuries (e.g. plastic reconstructive, orthopedic, and neurosurgeons with specialty in peripheral nerves) most typically repair or reconstruct a nerve that has been transected by creating an aligned coaptation of the two nerve endings and securing the separated nerve with nylon, polypropylene, or other monofilament sutures, fibrin glue, or combinations thereof. In some instances, an interposition element is applied between the two nerve endings (e.g. tube or graft), and such element is also secured with the same techniques. Depending on the location of the transection, it typically requires 3-18 months for complete nerve regeneration to occur and terminal functional recovery is often less than 50%. Furthermore, these results are highly variable and dependent on the surgeon's skills and technique used.

Both sutures and fibrin glue applied to the delicate nerve tissue at the area of nerve injury and regeneration have been associated with a local inflammatory response, which may lead to fibrosis and/or scarring of the healing nerve tissue due to foreign body reaction. Such fibrosis has been reported to inhibit the desired regenerative response and reduce and/or delay the functional recovery of the injured nerve.

Localized stresses created by overly tightened sutures applied at the neurorrhaphy site can further create excessive coaptation and bunching between the two nerve ends leading to nerve ‘strangulation’, which can limit blood flow leading to necrosis and/or additional scar tissue formation at the site of nerve repair, as well as axonal escape and neuroma formation, and/or excessive synkinesis. All of which prevent, delay, or limit nerve functional recovery.

For these and other reasons, there is a general need for methods and technologies to repair injured peripheral nerves without inducing the self-limiting inflammatory responses associated with the current surgical gold standard repair materials and techniques.

### SUMMARY

According to an aspect of the present inventive concepts, a system for connecting two nerve stumps comprises at least one neurorrhaphy device constructed and arranged to engage at least a portion of a proximal nerve stump and a distal nerve stump. The at least one neurorrhaphy device is configured to be deployed at one, two, or more deposit sites within a patient, and is further configured to provide a therapeutic benefit at the deposit site.

In some embodiments, the device is configured to align and/or reapproximate the proximal and distal nerve stumps. The alignment and/or reapproximation can be achieved without an application of sutures or other tissue penetrating components within, or in immediate proximity to, the proximal and/or distal nerve stumps.

In some embodiments, the device is configured to reduce iatrogenic trauma to the proximal and/or distal nerve stumps.

In some embodiments, the device is configured to improve functional recovery of the proximal and/or distal nerve stumps.

In some embodiments, the device is configured to improve the ease, reproducibility, and/or speed of connecting the proximal and distal nerve stumps.

In some embodiments, the device is configured to promote and/or otherwise support tissue growth proximate the deposit site.

In some embodiments, the deposit site comprises one, two, or more locations on and/or within a patient.

In some embodiments, the deposit site comprises a location within, around, and/or proximate a partial or full nerve transection. The deposit site can comprise a transected and repaired nerve.

In some embodiments, the device is configured to provide an interface between the proximal and distal nerve stumps.

In some embodiments, the device is configured to eliminate or otherwise reduce a gap length between the proximal and distal nerve stumps.

In some embodiments, the device is configured to maintain a calculated gap length between the proximal and distal nerve stumps. The calculated gap length can be configured to promote nerve cone sprouting and alignment between the proximal and distal nerve stumps.

In some embodiments, the device is configured to accommodate a nerve diameter of between 1 mm and 10 mm.

In some embodiments, the device comprises at least one longitudinal element configured to prevent or otherwise reduce a relative longitudinal shifting between the proximal and distal nerve stumps. The longitudinal element can comprise one, two, or more tension adjusting elements configured to adjust a tension applied to the proximal and distal nerve stumps by the longitudinal element. The longitudinal element and/or tension adjusting element can comprise one or more durable metals, alloys, and/or polymers. The longitudinal element and/or tension adjusting element can comprise one or more degradable metals, alloys, polymers, and/or biological materials. The longitudinal element can include one, two, or more elements selected from the group consisting of: cylindrical coil; cylindrical clam; cylindrical mesh obtained by braiding, knitting, or weaving; longitudinally and/or partially or completely circumferentially-interlocking elements; spacer between the proximal and distal nerve stumps; and combinations thereof.

In some embodiments, the device comprises at least one fixation element configured to prevent or otherwise reduce relative torsional shifting of the proximal and/or distal nerve stumps. The fixation element can be constructed and arranged to create one or more anchoring points proximate the proximal and/or distal nerve stumps. The fixation element can be anchored to a portion of the proximal and/or distal nerve stumps. The fixation element can be anchored to tissue proximate the proximal and/or distal nerve stumps. The fixation element can be anchored to organ tissue proximate the proximal and/or distal nerve stumps. The fixation element can further comprise an adhesive selected from the group consisting of: biological; mucus-based; fish glue; adhesive used in teeth whitening strip technology; adhesives used in oral mucosa bandages; and combinations thereof. Each fixation element can further comprise one, two, or more anchoring elements configured to physically tether or otherwise secure the fixation element to the proximal and/or distal nerve stump. The anchoring element can comprise an element selected from the group consisting of: plow tether; hook; clip; buttonhole; cringle; grommet; barb; adhesive; and combinations thereof. The fixation element and/or anchoring element can comprise one or more durable metals, alloys, and/or polymers. The fixation element and/or anchoring element can comprise one or more degradable metals, alloys, polymers, and/or biological materials. The fixation element and/or anchoring element can comprise one or more biologically derived materials, such as human amniotic membrane, blood vessels, umbilical cord vessels, and nerve tissue. The fixation element and/or anchoring element can comprise one or more synthetically derived materials, such as silicone, polyester or polyurethane plastics, and polytetrafluorethylene. The fixation element can comprise one, two, or more elements selected from the group consisting of: physical tether; circumferential clip; plow tether mounted via hinges secured to an annular structure; barbed hook; textured friction surface; adhesive element; elastic compression element; and combinations thereof. The fixation element can comprise an elastic metal, such as nitinol. The fixation element can comprise a super-elastic metal, such as super-elastic nitinol. The fixation element can comprise at least one surface comprising two or more projections configured to produce a variable internal diameter. The fixation element can comprise at least one surface comprising a texture configured to improve its frictional properties. The fixation element can comprise at least one surface comprising a texture configured to stimulate underlying nerve tissue via chemical treatments, abrasive texturization, treatments to create a sequential directional partial cutting texture, and/or other material removal strategies. The fixation element can comprise a helical coil. The first fixation element can comprise a right-handed pitch helical coil and the second fixation element can comprise a left-handed pitch helical coil. A clockwise rotation of the device about its longitudinal axis can cause the fixation elements to penetrate tissue proximate the proximal and distal nerve stumps. Progressive penetration of the fixation elements within the tissue can approximate and/or reduce tension between the proximal and distal nerve stumps. The fixation element can comprise a cuff.

In some embodiments, the device further comprises one, two, or more aligning elements configured to prevent or otherwise reduce relative alignment shifting of the proximal and/or distal nerve stumps. The aligning element can be configured to maintain the proximal and distal nerve stumps in an aligned geometry, a coapted geometry, and/or at a constant distance. The aligning element can be constructed and arranged to provide intrinsic mechanical protection to a coaptation site between the proximal and distal nerve stumps. The aligning element can be configured to prevent or otherwise reduce damage and/or loss of alignment between the proximal and distal nerve stumps. The aligning element can be configured to maintain a relative distance between the proximal and distal nerve stumps. The aligning element can comprise one or more durable metals, alloys, and/or polymers. The aligning element can comprise one or more degradable metals, alloys, polymers, and/or biological materials. The aligning element can be configured to surround at least a portion of the proximal and/or distal nerve stump. The aligning element can comprise an internal diameter similar to the external diameter of the proximal and/or distal nerve stump. The aligning element can comprise braided, knitted, or woven mesh. The mesh can be constructed from 2-0, 3-0, 4-0, 5-0, and/or 6-0 monofilament sutures. The mesh can be constructed by braiding 8, 16, 24, 32, or 48 suture threads in a 1:1, 1:2, or 2:2 relation. The mesh can comprise a braid angle of between 5 and 85 degrees, and the braid angle can define a braid pick per unit length.

In some embodiments, the system further comprises one, two, or more functional elements. The functional element can comprise an agent configured to prevent the formation of fibrotic tissue and/or neuroma at the deposit site. The functional element can comprise an anti-inflammatory agent configured to prevent or otherwise reduce inflammation and/or improve nerve remodeling at the deposit site. The functional element can comprise a lubricant. The functional element can comprise an adhesive. The functional element can be derived from decellularized extracellular matrix.

In some embodiments, one, two, or more components of the device are configured to degrade over time. The device component can be configured to degrade without eliciting an inflammatory response.

In some embodiments, the device is further configured to deliver a substance that induces, supports, and/or accelerates nerve regeneration.

In some embodiments, the device is further configured to deliver a substance that prevents the onset of negative responses.

In some embodiments, the device is further configured to provide visibility to the deposit site, such as to ensure correct longitudinal distancing and/or circumferential alignment between the proximal and distal nerve stumps.

In some embodiments, the device further comprises an external surface comprising one, two, or more lubricious materials.

In some embodiments, the device is configured to slide freely within tissue proximate the deposit site without generating mechanical resistance, irritation, inflammation, adhesion, and/or injury to the tissue,

In some embodiments, the device is configured to provide longitudinal stability, alignment stability, torsional stability, and/or additional features between the proximal and distal nerve stumps.

According to another aspect of the present inventive concepts, a neurorrhaphy device for connecting two nerve stumps comprises a first fixation element configured to engage tissue proximate a proximal nerve stump, a second fixation element configured to engage tissue proximate a distal nerve stump, and at least one aligning element extending between the first and second fixation elements.

In some embodiments, the aligning element is configured to surround at least a portion of the proximal and distal nerve stumps.

In some embodiments, the aligning element is mechanically coupled to the first and second fixation elements. The aligning element can comprise a cylindrical mesh configured to reduce its internal diameter when longitudinal tension and stretch is applied via the first and second fixation elements.

In some embodiments, the aligning element comprises a braided, knitted, or woven mesh constructed from threads made of one, two or more synthetic nondegradable and/or degradable materials.

In some embodiments, the aligning element comprises a braided, knitted, or woven mesh constructed from threads made of one, two or more biologically-derived materials.

In some embodiments, the aligning element comprises one, two, or more braided, knitted, and/or woven materials comprising a material selected from the group consisting of: biological degradable sutures, such as plain gut or chromic gut; biological non-degradable sutures, such as silk; synthetic degradable sutures, such PLLA, PLA, PGA, PCA, PLLA-PGA; non degradable sutures, such as nylon, polypropylene, PTFE, PET, and PETG; and combinations thereof.

In some embodiments, the aligning element comprises a braided, knitted, or woven mesh constructed from a plurality of monofilament threads obtained from extracellular matrices. The monofilament threads can comprise an extracellular matrix obtained from a series of mechanical and/or chemical treatments. The monofilament threads can be configured to alter one, two, or more of its properties upon rehydration, thereby resulting in a structural and/or chemical change to the threads.

In some embodiments, the fixation elements comprise a helical coil configured to concentrically surround at least a portion of the proximal and distal nerve stumps.

In some embodiments, the fixation elements further comprise one, two, or more anchoring elements. The anchoring element can be configured to penetrate, indent, or otherwise interfere with a surface of the nerve stump. The anchoring element can comprise a unidirectional barbed hook.

In some embodiments, the aligning element comprises a braided, knitted, or woven mesh constructed with threads configured to be longitudinally compressed prior to insertion of the proximal and distal nerve stumps. The aligning element can be configured to slidingly receive at least a portion of the proximal and distal nerve stumps. The compressed aligning element can comprise an inner diameter that is larger than a diameter of the proximal and distal nerve stumps.

In some embodiments, the aligning element comprises a braided cuff constructed from 8 or 16 threads of 5-0 monofilament suture and is further constructed in a 1:1 or 2:2 relation. The cuff can comprise a porosity of between 20% and 60%. The cuff can comprise a picks per inch of between 10 and 40. The cuff can comprise a braid angle of between 25% and 35%.

According to another aspect of the present inventive concepts, a tool for deploying a neurorrhaphy device comprises at least two arms controlled by a set of handles. The tool is constructed and arranged to hold a preloaded neurorrhaphy device in a compressed state. The tool is constructed and arranged to release the neurorrhaphy device from the compressed state.

In some embodiments, the tool comprises three extendable arms comprising two movable arms and one static arm.

In some embodiments, the tool further comprises a release trigger. The trigger can be constructed within the set of handles.

In some embodiments, the tool further comprises a housing configured to slidingly receive at least a portion of the neurorrhaphy device.

According to another aspect of the present inventive concepts, a system for testing functional characteristics of a neurorrhaphy device comprises a uniaxial testing apparatus, a visual recording device, a device support, a nerve support, and/or a field of view background. The system is constructed and arranged to test one or more dynamic responses of the neurorrhaphy device.

In some embodiments, the apparatus comprises a programmable motorized linear stage controlled by a digital controller, actuated by servo or stepper motors with sensors collecting real-time position and displacement data, and a digital force gauge mounted to the motorized linear stage collecting real-time force data.

In some embodiments, the visual recording device comprises a high-definition video camera.

In some embodiments, the device support comprises one, two, or more textured cylindrical posts comprising a diameter similar to an inner diameter of the neurorrhaphy device. The device support can be mounted along a central axis of the apparatus. A first device support can be mounted to a force gauge of the apparatus and a second device support can be mounted to a base plate of the apparatus.

In some embodiments, the nerve support comprises one, two, or more textured, adjustable vice or chuck grips. The nerve support can be mounted along a central axis of the apparatus. A first nerve support can be mounted to a force gauge of the apparatus and a second nerve support can be mounted to a base plate of the apparatus.

In some embodiments, the field of view background is positioned planarly with a central axis of the apparatus and/or neurorrhaphy device.

According to another aspect of the present inventive concepts, a system for connecting two nerve stumps comprises a neurorrhaphy device. The neurorrhaphy device comprises: a first anchoring element for attachment to a proximal nerve stump (e.g. at a location away from the end of the proximal nerve stump); a second anchoring element for attachment to a distal nerve stump (e.g. at a location away from the end of the distal nerve stump); a longitudinal element for connection between the first anchoring element and the second anchoring element. The system can avoid the use of sutures, surgical clips, or glues, and other similar components penetrating in or used with nerve stumps.

The technology described herein, along with the attributes and attendant advantages thereof, will best be appreciated and understood in view of the following detailed description taken in conjunction with the accompanying drawings in which representative embodiments are described by way of example.

### INCORPORATION BY REFERENCE

All publications, patents, and patent applications mentioned in this specification are herein incorporated by reference to the same extent as if each individual publication, patent, or patent application was specifically and individually indicated to be incorporated by reference. The content of all publications, patents, and patent applications mentioned in this specification are herein incorporated by reference in their entirety for all purposes.

### BRIEF DESCRIPTION OF THE DRAWINGS

FIGS. 1 and 1A-D illustrate a schematic view of a system comprising a neurorrhaphy device and side-sectional views of a nerve transection, consistent with the present inventive concepts.

FIG. 2 illustrates an embodiment of a neurorrhaphy device, consistent with the present inventive concepts.

FIG. 3 illustrates an embodiment of a neurorrhaphy device, consistent with the present inventive concepts

FIG. 4 illustrates an embodiment of a neurorrhaphy device, consistent with the present inventive concepts.

FIGS. 5A and 5B illustrate an embodiment of a neurorrhaphy device, consistent with the present inventive concepts.

FIGS. 6A and 6B illustrate an embodiment of a neurorrhaphy device, consistent with the present inventive concepts.

FIGS. 7A-C illustrate an embodiment of a neurorrhaphy device, consistent with the present inventive concepts.

FIG. 8 illustrates an embodiment of a neurorrhaphy device, consistent with the present inventive concepts.

FIG. 9 illustrates an embodiment of a neurorrhaphy device, consistent with the present inventive concepts.

FIGS. 10A-D illustrate an embodiment of a neurorrhaphy device, consistent with the present inventive concepts.

FIGS. 11A and 11B illustrate an embodiment of a nerve fixation element including anchoring elements, consistent with the present inventive concepts.

FIGS. 12A and 12B illustrate an embodiment of a neurorrhaphy device, consistent with the present inventive concepts.

FIGS. 13A and 13B illustrate an embodiment of a neurorrhaphy device, consistent with the present inventive concepts.

FIGS. 14A-D illustrate an embodiment of a neurorrhaphy device, consistent with the present inventive concepts.

FIGS. 15A and 15B illustrate an embodiment of a neurorrhaphy device, consistent with the present inventive concepts.

FIGS. 16A and 16B illustrate an embodiment of a neurorrhaphy device, consistent with the present inventive concepts.

FIGS. 17A and 17B illustrate an embodiment of a neurorrhaphy device, consistent with the present inventive concepts.

FIG. 18 illustrates an embodiment of a neurorrhaphy device, consistent with the present inventive concepts.

FIGS. 19A and 19B, illustrate an embodiment of a neurorrhaphy device, consistent with the present inventive concepts.

FIGS. 20A and 20B, illustrate an embodiment of a neurorrhaphy device, consistent with the present inventive concepts.

FIGS. 21A and 21B, illustrate an embodiment of a neurorrhaphy device, consistent with the present inventive concepts.

FIGS. 22A and 22B, illustrate an embodiment of a neurorrhaphy device, consistent with the present inventive concepts.

FIGS. 23A-C illustrate an embodiment of a neurorrhaphy device, consistent with the present inventive concepts.

FIGS. 24A and 24B, illustrate an embodiment of a neurorrhaphy device, consistent with the present inventive concepts.

FIGS. 25A-C illustrate an embodiment of a neurorrhaphy device, consistent with the present inventive concepts.

FIGS. 26A and 26B, illustrate an embodiment of a neurorrhaphy device, consistent with the present inventive concepts.

FIGS. 27A-C illustrate an embodiment of a neurorrhaphy device, consistent with the present inventive concepts.

FIGS. 28A-C illustrate an embodiment of a neurorrhaphy device, consistent with the present inventive concepts.

FIGS. 29A and 29B, illustrate an embodiment of a neurorrhaphy device, consistent with the present inventive concepts.

FIGS. 30A and 30B, illustrate an embodiment of a neurorrhaphy device, consistent with the present inventive concepts.

FIGS. 31A and 31B, illustrate an embodiment of a neurorrhaphy device, consistent with the present inventive concepts.

FIGS. 32A-E illustrate an embodiment of a tool for holding and/or deploying a neurorrhaphy device, consistent with the present inventive concepts.

FIGS. 33A-E illustrate an embodiment of a tool for holding and/or deploying a neurorrhaphy device, consistent with the present inventive concepts.

FIG. 34 thru 37A and 37B illustrate an embodiment of a system for testing and/or verifying functional characteristics of a neurorrhaphy device in vitro and example output data, consistent with the present inventive concepts.

FIGS. 38A and 38B, illustrate an embodiment of tool for testing and/or verifying functional characteristics of a neurorrhaphy device ex vivo, consistent with the present inventive concepts.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, and so on may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” and the like).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, and “prevention” shall include the acts of “reduce”, “reducing”, and “reduction”, respectively.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereof” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A; one, two, three or more of item B; and/or one, two, three, or more of item C.

In this specification, unless explicitly stated otherwise, “and” can mean “or”, and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

As used herein, when a quantifiable parameter is described as having a value “between” a first value X and a second value Y, it shall include the parameter having a value of: at least X, no more than Y, and/or at least X and no more than Y. For example, a length of between 1 and 10 shall include a length of at least 1 (including values greater than 10), a length of less than 10 (including values less than 1), and/or values greater than 1 and less than 10.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

The terms “major axis” and “minor axis” of a component where used herein are the length and diameter, respectively, of the smallest volume hypothetical cylinder which can completely surround the component.

As used herein, the term “functional element” is to be taken to include one or more elements constructed and arranged to perform a function. A functional element can comprise a sensor and/or a transducer. In some embodiments, a functional element is configured to deliver energy and/or otherwise treat tissue (e.g. a functional element configured as a treatment element). Alternatively or additionally, a functional element (e.g. a functional element comprising a sensor) can be configured to record one or more parameters, such as a patient physiologic parameter; a patient anatomical parameter (e.g. a tissue geometry parameter); a patient environment parameter; and/or a system parameter. In some embodiments, a sensor or other functional element is configured to perform a diagnostic function (e.g. to gather data used to perform a diagnosis). In some embodiments, a functional element is configured to perform a therapeutic function (e.g. to deliver therapeutic energy and/or a therapeutic agent). In some embodiments, a functional element comprises one or more elements constructed and arranged to perform a function selected from the group consisting of: deliver energy; extract energy (e.g. to cool a component); deliver a drug or other agent; manipulate a system component or patient tissue; record or otherwise sense a parameter such as a patient physiologic parameter or a system parameter; and combinations of one or more of these. A functional element can comprise a fluid and/or a fluid delivery system. A functional element can comprise a reservoir, such as an expandable balloon or other fluid-maintaining reservoir. A “functional assembly” can comprise an assembly constructed and arranged to perform a function, such as a diagnostic and/or therapeutic function. A functional assembly can comprise an expandable assembly. A functional assembly can comprise one or more functional elements.

The term “transducer” where used herein is to be taken to include any component or combination of components that receives energy or any input, and produces an output. For example, a transducer can include an electrode that receives electrical energy, and distributes the electrical energy to tissue (e.g. based on the size of the electrode). In some configurations, a transducer converts an electrical signal into any output, such as: light (e.g. a transducer comprising a light emitting diode or light bulb), sound (e.g. a transducer comprising a piezo crystal configured to deliver ultrasound energy); pressure (e.g. an applied pressure or force); heat energy; cryogenic energy; chemical energy; mechanical energy (e.g. a transducer comprising a motor or a solenoid); magnetic energy; and/or a different electrical signal (e.g. different than the input signal to the transducer). Alternatively or additionally, a transducer can convert a physical quantity (e.g. variations in a physical quantity) into an electrical signal. A transducer can include any component that delivers energy and/or an agent to tissue, such as a transducer configured to deliver one or more of: electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); chemical energy; electromagnetic energy; magnetic energy; and combinations of one or more of these.

As used herein, the term “fluid” can refer to a liquid, gas, gel, or any flowable material, such as a material which can be propelled through a lumen and/or opening.

As used herein, the term “material” can refer to a single material, or a combination of two, three, four, or more materials.

Figures and/or relative dimensions of components represented within figures as referred to herein may not be to scale.

It is appreciated that certain features of the inventive concepts, which are, for clarity, described in the context of separate embodiments, may also be provided in combination in a single embodiment. Conversely, various features of the inventive concepts which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the inventive concepts have been simplified to focus on elements that are relevant for a clear understanding of the inventive concepts, while eliminating, for purposes of clarity, other elements that those of ordinary skill in the art will appreciate may also comprise a portion of the inventive concepts. However, because such elements are well known in the art, and because they do not necessarily facilitate a better understanding of the inventive concepts, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

Embodiments of the systems, devices and methods described herein can be directed to systems, devices, and methods for performing a procedure including the alignment and reapproximation of nerve segments, such as a procedure performed without the need for applying sutures or other components (e.g. tissue penetrating components) within, or in immediate proximity to, the area where the nerve injury (e.g. transection) has occurred. The inventive concepts as described herein are intended to: reduce the iatrogenic trauma made to peripheral nerves during nerve repair procedures; improve the functional recovery following nerve repair; improve the ease, reproducibility, and/or speed of the nerve repair procedure; and/or reduce the difference in outcome to variability of surgical techniques and skills used.

Embodiments of the systems, devices, and methods described herein can be adapted for use with systems, devices, and methods for performing a procedure including the alignment and reapproximation of other anatomical tubular or cylindrical structures including, but not limited to: blood vessels; lymphatic vessels; gastrointestinal tubular structures; osteomuscular cylindrical structures, such as tendons, ligaments, and muscular bundles; and genitourinary tubular structures, such as the ureter and urethra. These embodiments can be employed to prevent or otherwise reduce the need for applying sutures or other tissue penetrating components within, or in immediate proximity to, the area where the specific tissue reapproximation is needed. These embodiments can be intended to: reduce the iatrogenic trauma made to cylindrical and/or tubular anatomical structures during repair procedures; improve the ease, reproducibility, and/or speed of the repair procedure; improve the functional recovery following repair; and/or reduce the difference in outcome to variability of surgical techniques and skills used.

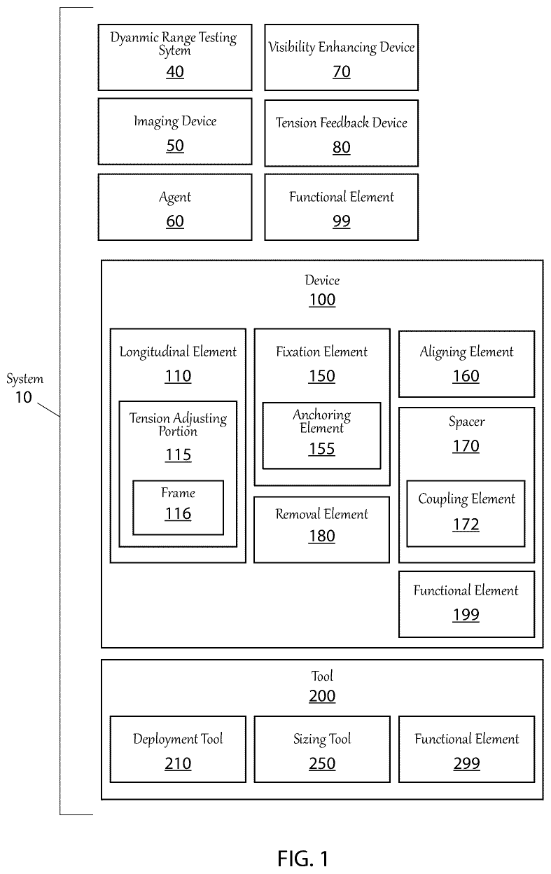

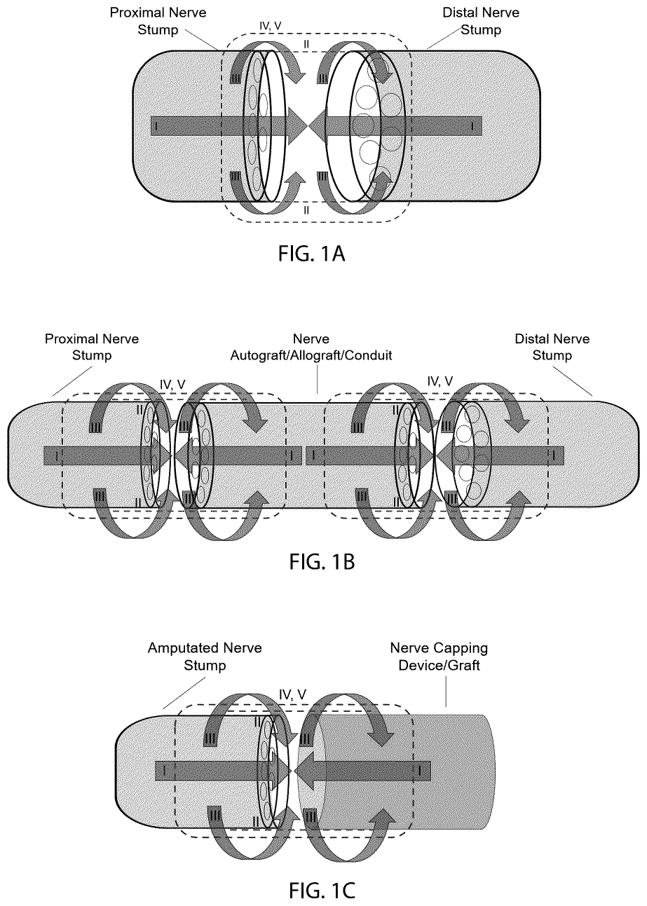

Referring now to FIGS. 1 and 1A-C, a schematic view of a system comprising a neurorrhaphy device and side-sectional views of a nerve transection are illustrated, respectively, consistent with the present inventive concepts.

Referring specifically to FIG. 1, system 10 comprises a neurorrhaphy device, device 100 shown, as well as various components for manufacture and/or deployment of device 100. Device 100 is configured to be deployed (e.g. inserted, delivered, implanted, and the like) at one, two, or more “deposit sites”, such as to provide a therapeutic benefit at the deposit site. Device 100 can be deployed at the deposit site to promote, and/or otherwise support, tissue growth of a patient (e.g. support nerve and/or other tissue growth and/or regeneration at locations proximate and/or remote from the deposit site). In some embodiments, device 100 remodels over time (e.g. into native tissue of the patient). As used herein, the deposit site can comprise one, two, or more locations on and/or within the patient, and device 100, as referred to herein, can comprise one, two, or more devices 100.

As described herein, the deposit site can comprise a location within, and/or around, and/or proximate a partial or full nerve transection, such as a transected and repaired nerve (e.g. to be treated via an epineural and/or fascicular repair, such as neurorrhaphy). For example, device 100 can be deployed to provide an interface between two or more nerves or nerve stumps. The two or more nerve stumps can be coapted (e.g. directly) to eliminate or otherwise reduce a gap length between the nerve stumps. Alternatively, the two or more nerve stumps are not coapted and a calculated gap length is maintained between the nerve stumps. The calculated gap length can be configured to promote nerve cone sprouting and alignment from a proximal nerve stump having a greater degree of freedom to properly align toward a distal nerve stump.

Device 100 can be configured to accommodate different nerve diameters, such as a nerve diameter of between 1 mm and 10 mm (e.g. 1 mm or more, and/or 10 mm or less). In some embodiments, at least one component of device 100 is adaptable to the nerve stump diameter. In some embodiments, device 100 is available in multiple different diameters to allow a clinician (e.g. surgeon) to select the most appropriate size for the nerve stump, such as a set of devices 100 constructed and arranged to accommodate a set of nerve diameters, such as nerve diameters of 1-2 mm, 2-3 mm, 3-4 mm, 4-5 mm, 5-6 mm, or 6-7 mm. The clinician can utilize one, two, or more tools 200 to select an appropriately sized device 100, such as deployment tool 210 and/or sizing tool 250 described herein.

Device 100 can comprise one, two, or more longitudinal elements and/or other elongate structure, longitudinal element 110 shown. Longitudinal element 110 can be configured to prevent or otherwise reduce relative longitudinal shifting of the proximal and distal nerve stumps being coapted by device 100. In some embodiments, longitudinal element 110 comprises one, two, or more elements configured to adjust the tension applied by longitudinal element 110, tension adjusting element 115 shown. Longitudinal element 110 and/or tension adjusting element 115 can be coupled (e.g. mechanically coupled) to one or more components of device 100 (e.g. fixation element 150, aligning element 160, and the like). Longitudinal element 110 and/or tension adjusting element 115 can comprise one or more durable (e.g. metals, alloys, polymers, and the like) and/or degradable (e.g. biodegradable metals, alloys, polymer, biological materials, and the like) materials.

Longitudinal element 110 can include one, two, or more elements selected from the group consisting of: cylindrical coil; cylindrical clam; cylindrical mesh obtained by braiding, knitting, or weaving; longitudinally and/or partially or completely circumferentially-interlocking elements; spacer between two nerve stumps; and combinations of these.

Longitudinal element 110 can be constructed and arranged as a clam design, such as a structure including one, two, or more cylindrical interdigitation elements configured to allow a temporary disruption of the cylindrical continuity of the cylindrical structure by retracting the interdigitation elements around a longitudinal hinge and/or axis to allow application of such structure around another cylindrical structure.

Longitudinal element 110 can include multiple interlocking segments configured to alter the length of device 100 to fit a coaptation site. In some embodiments, one or more of the interlocking segments are attached to a fixation element 150 (described hereinbelow) and can further include predefined visual markings indicating a minimum distance fixation element 150 can be inserted into the surrounding tissue to provide sufficient holding strength and/or approximation of the nerve stumps.

Device 100 can comprise one, two, or more fixation elements, fixation element 150 shown. Fixation element 150 can comprise one or more components singly or collectively configured to prevent or otherwise reduce relative torsional shifting of the proximal and/or distal nerve stumps. Fixation element 150 can be constructed and arranged to create one or more anchoring points in a location and manner that is non-traumatic for the nerve's internal structure (e.g. fascicles). In some embodiments, a fixation element 150 is anchored to a portion of the proximal or distal nerve stump. In some embodiments, a fixation element 150 is anchored to tissue proximate the proximal or distal nerve stump. In some embodiments, a fixation element 150 is anchored to bone proximate the proximate or distal nerve stump. In some embodiments, a fixation element 150 is anchored to organ tissue proximate the proximal or distal nerve stump. Fixation element 150 can comprise an adhesive selected from the group consisting of: biological; mucus-based; fish glue; adhesive used in teeth whitening strip technology; adhesives used in oral mucosa bandages; and combinations of these. In some embodiments, fixation element 150 comprises one, two, or more anchoring elements, anchoring element 155 shown, which can be configured to attach (e.g. physically tether or otherwise secure) fixation element 150 to a nerve stump. Anchoring element 155 can comprise an element selected from the group consisting of: plow tethers; hooks; clips; buttonholes; cringles; grommets; barbs; adhesives; and combinations of these. Fixation element 150 and/or anchoring element 155 can comprise one or more durable (e.g. metals, alloys, polymers, and the like) and/or degradable (e.g. biodegradable metals, alloys, polymer, biological materials, and the like) materials. Fixation element 150 and/or anchoring element 155 can comprise one or more biologically derived materials, such as human amniotic membrane, blood vessels, umbilical cord vessels, and nerve tissue. Fixation element 150 and/or anchoring element 155 can comprise one or more synthetically derived materials, such as silicone, polyester or polyurethane plastics, and polytetrafluorethylene.

Fixation element 150 can comprise one, two, or more elements selected from the group consisting of: physical tether (e.g. one or more helical coils); circumferential clip; plow tether mounted via hinges secured to an annular structure; barbed hook; textured friction surface; adhesive element; elastic compression element; and combinations of these.

Fixation element 150 can comprise an elastic metal, such as nitinol. Fixation element 150 can comprise a super-elastic metal, such as super-elastic nitinol.

At least one surface of fixation element 150 can comprise two or more projections (e.g. spikes), such as to produce a variable internal diameter. At least one surface of fixation element 150 can comprise a texture configured to improve its frictional properties. At least one surface of fixation element 150 can comprise a texture configured to stimulate underlying nerve tissue via chemical treatments, abrasive texturization, treatments to create a sequential directional partial cutting texture (e.g. fish scale texture), and/or other material removal strategies.

As shown in Table 1 below, device 100 can provide numerous combinations for the connections of longitudinal element 110 and fixation element 150 at one or more locations proximate the transected nerve.

TABLE 1
Combinations of longitudinal element 110
and fixation element 150 connections

| | | Distal Nerve | Both Distal Nerve |
|---|---|---|---|
| | Distal Nerve | Surrounding | And Distal |
| | Stump Alone | Tissue Alone | Surrounding Tissue |
| Proximal Nerve | 1 | 4 | 8 |
| Stump Alone | | | |
| Proximal Nerve | 5 | 2 | 9 |
| Surrounding Tissue | | | |
| Alone | | | |
| Both Proximal Nerve | 6 | 7 | 3 |
| And Proximal | | | |
| Surrounding Tissue | | | |

Device 100 can comprise one, two, or more aligning elements, aligning element 160 shown. Aligning element 160 can be configured to prevent or otherwise reduce relative alignment shifting of the proximal and distal nerve stumps. In some embodiments, and as described herein, aligning element 160 can be constructed and arranged to replace or supplement the function of longitudinal element 110. Aligning element 160 can be configured to maintain the proximal and distal nerve stumps: in an aligned geometry; in a coapted geometry; at a constant distance; and/or a combination of two or all three of these. Aligning element 160 can be constructed and arranged to provide intrinsic mechanical protection to the nerve coaptation site. In some embodiments, aligning element 160 is configured to prevent or otherwise reduce damage and/or loss of alignment, and/or to maintain the relative distance between the proximal and distal nerve stumps (e.g. by resisting stretching, bending, slippage, and the like). Aligning element 160 can comprise one or more durable (e.g. metals, alloys, polymers, and the like) and/or degradable (e.g. biodegradable metals, alloys, polymer, biological materials, and the like) materials.

Aligning element 160 can comprise one, two, or more configurations of longitudinal element 110 and/or fixation element 150.

One, two, or more components of longitudinal element 110, fixation 150, and/or aligning element 160 can comprise a material selected from the group consisting of: metals (e.g. NiTi in super-elastic and/or memory shape state, NiTi type beta and similar alloys, stainless steels like 316L or 304, and/or degradable magnesium alloys); synthetic degradable and nondegradable polymers (e.g. PTFE, polyesters, polyurethanes, polyamides, and/or other polymers); biologically derived materials (e.g. collagen, elastin, proteoglycans, glycosaminoglycans, and/or extracellular matrix derived materials); and combinations of these.

Device 100 can comprise one, two, or more space occupying components, spacer 170 shown. Spacer 170 can be configured to impose a defined distance between the proximal and distal nerve stumps. In some embodiments, the define distance approaches 0 and the nerve stumps are coapted together. Spacer 170 can be configured to support directional neurite migration (e.g. support the nerve regeneration process) from the proximal stump toward the distal stump, such that spacer 170 is permeable to nerve regeneration components including cells, axons, blood vessels, and the like. In some embodiments, spacer 170 is configured to guide neurites from the proximal nerve stump toward the distal nerve dump. Spacer 170 can comprise one or more components that are constructed and arranged to provide permeability (e.g. selective) to the nerve coaptation site. Spacer 170 can comprise a mesh density that is controlled via one, two, or more textile manufacturing techniques configured to provide selective permeability. In some embodiments, spacer 170 allows for oxygen and/or nutrients to permeate from surrounding tissue to the nerve coaptation site. In some embodiments, spacer 170 can be impermeable to neurites, such as to prevent the escape of such neurites out of the boundaries of the nerve epineurium (e.g. to prevent neuroma formation). In some embodiments, spacer 170 provides a barrier for inflammatory cells to migrate from surrounding tissue to the nerve coaptation site. In some embodiments, spacer 170 provides a barrier to prevent the infiltration of fibroblasts and/or the formation of fibrotic tissue from surrounding tissue to the nerve coaptation site. Spacer 170 can comprise one or more durable (e.g. metals, alloys, polymers, and the like) and/or degradable (e.g. biodegradable metals, alloys, polymer, biological materials, and the like) materials. Spacer 170 can be constructed and arranged to comprise a cylindrical volume between the proximal and distal nerve stumps, such that spacer 170 can be filled with air, saline, and/or other fluid.

Spacer 170 can comprise one, two, or more biologically derived materials, such as an extracellular matrix. Spacer 170 can comprise one, two, or more synthetic materials.

Device 100 can comprise one, two, or more component removal elements, removal element 180 shown. Removal element 180 can be constructed and arranged to allow for the immediate removal of device 100 due to incorrect placement or deployment. In some embodiments, removal element 180 comprises a longitudinal split line configured to be easily cut with surgical scissors in order to split device 100 into two or more removable portions. In some embodiments, device 100 comprises a woven fabric and removal element 180 comprises one or more fabric threads (e.g. braided, knitted, and/or woven fabric threads) configured to be removed in order to split device 100 into two or more removable portions. Removal element 180 can comprise one or more durable (e.g. metals, alloys, polymers, and the like) and/or degradable (e.g. biodegradable metals, alloys, polymer, biological materials, and the like) materials.

Device 100 can comprise one, two, or more functional elements, functional element 199 shown. Functional element 199 can comprise an agent configured to be released, over time, and configured to foster and/or prevent certain biological phenomena at the deposit site. In some embodiments, functional element 199 comprises a growth factor configured to accelerate the nerve repair process. In some embodiments, functional element 199 comprises an immunomodulatory factor configured to foster a beneficial inflammatory response during the nerve repair process. In some embodiments, functional element 199 comprises an antimicrobial agent configured to prevent an onset of infection at the deposit site. In some embodiments, functional element 199 comprises an agent configured to prevent the formation of fibrotic tissue and/or neuroma at the deposit site. In some embodiments, functional element 199 comprises an anti-inflammatory agent configured to prevent or otherwise reduce inflammation and/or improve nerve remodeling at the deposit site. In some embodiments, functional element 199 comprises a lubricant. In some embodiments, functional element 199 comprises an adhesive. In some embodiments, functional element 199 comprises one, two, or more elements derived from decellularized extracellular matrix. In some embodiments, functional element 199 comprises a calcineurin inhibitor, such as tacrolimus or FK506. In some embodiments, functional element 199 comprises an anticonvulsant, such as gabapentin. In some embodiments functional element 199 comprises a pharmaceutical composition configured to reduce neuropathic pain and/or improve nerve healing.

In some embodiments, device 100 allows for two severed nerve stumps to be reapproximated at a desired distance without use of suture or other similar component interfering in the coaptation area.

In some embodiments, device 100 provides longitudinal stability (e.g. prevents the proximal and distal nerve stumps from changing their mutual distance in either direction) to the nerve stumps. Longitudinal stability can prevent bunching, strangulation by compression, and/or tearing by extension, of the nerve stumps.

In some embodiments, device 100 provides torsional stability to the two nerve stumps.

In some embodiments, device 100 maintains alignment of the two nerve stumps at a constant distance.

In some embodiments, device 100 provides stability and/or alignment over a sufficient period of time for the nerve to self-support and heal.

In some embodiments, device 100 is biocompatible and non-inflammatory, sterile, disposable (e.g. single use), and pyrogen free.

In some embodiments, device 100 is contained within a sterile barrier packaging that is stored in one, two, or more defined environmental conditions.

In some embodiments, device 100 allows for one or more bending motions consistent with anatomical forces found in the repair locations, for example without allowing kinks.

In some embodiments, device 100 provides consistent and reproducible results (e.g. efficacy and/or other therapeutic results).

In some embodiments, device 100 provides mechanical protection around the nerve coaptation site, such that device 100 can prevent or otherwise reduce damage and/or loss of alignment and maintain a relative distance between two nerve stumps by resisting stretch, bending, slippage, etc. Device 100 can be configured to provide mechanical protection against nerve compression, extension, torsion, and/or bending. In some embodiments, device 100 provides other types of protection to the nerve coaptation, such as allowing selected permeability to, or exclusion of, certain cellular elements.

In some embodiments, one, two, or more components of device 100 degrade (e.g. biodegrade) over time. One, two, or more components of device 100 can be configured to degrade without eliciting an inflammatory response, such as a fibrotic and/or scarring response.

In some embodiments, device 100 provides for the delivery of substances that induce, support, and/or accelerate nerve regeneration and/or prevent the onset of negative responses.

In some embodiments, device 100 provides visibility to the site of a nerve coaptation, such as to ensure correct longitudinal distancing and/or circumferential alignment between the nerve stumps and/or nerve fascicles.

In some embodiments, device 100 comprises an external surface comprising one, two, or more lubricious materials. Device 100 can be configured to slide freely within the surrounding tissue, such as during the normal body movement, without generating mechanical resistance, irritation, inflammation, adhesions, and/or injury to the surrounding tissue.

In some embodiments, device 100 is configured for application within a direct nerve repair, such as a deep sharp injury and/or laceration resulting in one or more nerves being transected. Direct nerve repair applications can require a high level of longitudinal tension applied to an end-to-end repair thereby reconnecting the two nerve stumps (e.g. neurorrhaphy). Direct nerve repair applications includes intact native nerves that are normally under longitudinal tensions and, in some instances, further includes nerve trimming necessary to create adequate coaptation surfaces leading to additional levels of longitudinal tension. Longitudinal tension can be supported by the discrete number of sutures used for the primary repair. However, these sutures, normally applied to the epineurium of the two nerve stumps, create significant localized stresses on the nerve tissue supporting them. Significant localized stresses not only elicit the formation of fibrotic tissue as a normal tissue-reinforcing/remodeling adaptive response, but also prevent local microvascular perfusion, which can contribute to creating a significant inflammatory and fibrotic and/or scarring response (e.g. commonly observed around the sutures). The formation of fibrotic tissue and/or prevention of local microvascular perfusion can be compounded by the foreign body response elicited by the suture material itself, such as a suture material comprising non-degradable nylon or polypropylene monofilaments. As described herein, device 100 can reduce one, two, or more of these described elements that can induce fibrosis. For example, device 100 can reduce localized stresses by distributing the localized longitudinal stress over larger surfaces and/or away from the delicate nerve epineural tissue. Distributing localized longitudinal stress over larger surfaces and/or away from delicate nerve epineural tissue can mitigate both the remodeling response and the microcirculatory injury. Additionally, as described herein, device 100 can mitigate a foreign body response by using degradable materials known for reducing and/or eliminating one or more fibrotic foreign body responses.

In some embodiments, device 100 is configured for application within a nerve transfer procedure. Nerve transfers can be required when severe (e.g. axonotmesis and neurotmesis) proximal (e.g. toward the spinal root ganglia) nerve injuries occur. Local nerve repair may create an intact path for axons to sprout, regrow, and/or extend from the location of the proximal injury to the distal functional component (e.g. neuromuscular junctions, sensory cells, etc.). However, the extensive time required for the axons to regrow can lead to irreversible atrophy in the distal functional components thereby preventing functional recovery. For this reason, severe proximal nerve injuries are commonly treated with a nerve transfer, as opposed to a primary repair. Nerve transfers can include the redirection of a portion of a healthy distal nerve and its connection to a functional element (e.g. a muscle) that was originally downstream to the injured nerve. Connection between the healthy distal nerve and functional element can be made via an end-to-end neurorrhaphy (e.g. nerve coaptation often using two or more monofilament synthetic sutures) between the redirected portion of the healthy nerve and a free nerve pedicle obtained by resecting the injured nerve in proximity to the muscle that it innervates. The level of longitudinal tensions at the nerve transfer can be insignificant because of the geometrical configuration of the repair, however, higher curvatures may be experienced when a secure attachment of the two nerve stumps is desirable. As described herein, device 100 can mitigate the inflammatory response derived from the foreign body reaction to the sutures commonly used for nerve transfer procedures.

In some embodiments, device 100 is configured for application within a nerve cable graft. Larger diameter nerve gap defects caused by acute or iatrogenic injury, and resulting nerve tissue loss, are commonly repaired with a cable graft approach. Cable grafts comprise multiple segments of a smaller caliber nerve used in parallel to bridge a larger diameter nerve defect. Cable grafts utilize a great amount of suture material to connect each individual nerve segment at the two ends of the defect, thereby resulting in an aggravation of the foreign body response. As described herein, device 100 can bridge the full length of the defect with a sufficient overlap with both the proximal and distal stump. Additionally, device 100 can arrange the multiple parallel nerve segments in a cylindrical configuration, thereby facilitating and/or accelerating the in vivo remodeling of the multiple nerve segments into a cohesive single nerve bridge between the two nerve stumps.

System 10 can comprise one, two, or more imaging devices, imaging device 50 shown.

System 10 can comprise one, two, or more pharmaceutical drugs or other agents, agent 60 shown.

System 10 can comprise one, two, or more visibility enhancing components, visibility enhancing devices 70 shown. Visibility enhancing device 70 can comprise one or more components that are singly or collectively configured to improve the ability of the clinician to visualize the field of view, working area, and/or device 100. In some embodiments, visibility enhancing device 70 is selected from the group consisting of: a high-contrast background material/sheet (e.g. with a color of blue, yellow, green, and the like) to be placed downstream to the field of view and/or working area; a translucent, diffractive, or reflective material configured to improve the propagation of the external surgical lighting; a high-contrast color applied to at least a portion of device 100; a path where light can be channeled (either external surgical light, or light from a dedicated fiber optic illuminator) and used to visualize and/or illuminate the field of view and/or working area; and combinations of these.

System 10 can comprise one, two, or more components to provide tension information, tension feedback device 80 shown. Tension feedback device 80 can be configured to inform the clinician via visual, tactile, and/or other feedback mechanisms when a desired and/or excessive tension is achieved while reapproximating the nerve endings. In some embodiments, if excessive tension is sensed and/or otherwise determined, an interpositional element can be deployed between the proximal and distal nerve stumps.

System 10 can comprise one, two, or more functional elements, functional element 99 shown.

System 10 can comprise one, two, or more tools, tool 200 shown, which can comprise a tool configured to aid in the deployment, positioning, and/or removal of device 100 at the deposit site. In some embodiments, tool 200 comprises deployment tool 210. Deployment tool 210 can comprise a disposable or reusable tool (e.g. a disposable or reusable surgical tool provided in a sterile state within a sterile barrier packaging that is stored in one, two, or more defined environmental conditions). Deployment tool 210 can be loaded (e.g. pre-loaded) with one or more devices 100. Deployment tool 210 can be constructed and arranged to enable suitable surgical access and tool orientation into the surgical cavity where the nerve repair procedure is performed, such as to enable access to restrictive cavities around digital nerves. Deployment tool 210 can be constructed and arranged to allow controlled and reproducible implantation and positioning of device 100, such as by manipulating: the relative rotational alignment between the proximal and distal nerve stump; the distance between the proximal and distal nerve stumps; the location and anchoring of device 100 at the peripheries of the proximal and distal nerve stumps; the activation of one or more components (e.g. shape memory components) of device 100; and the internal diameter of aligning element 160. Deployment tool 210 can be constructed and arranged to measure the diameter of the proximal and/or distal nerve stump, so as to inform the clinician of the appropriately sized device 100 to deploy. Deployment tool 210 can be constructed and arranged to measure the distance between the proximal and distal never stumps, such as to inform the clinician of the appropriate placement of one or more fixation elements 150. In some embodiments, a medical-grade lubricant (e.g. silicone) can be used in conjunction with deployment tool 210 at the interface between deployment tool 210 and device 100, and/or in other areas of deployment tool 210. In some embodiments, tool 200 comprises sizing tool 250. Sizing tool 250 can comprise a disposable or reusable tool (e.g. provided in a sterile state within a sterile barrier packaging that is stored in one, two, or more defined environmental conditions). Sizing tool 250 can be constructed and arranged to measure the diameter of the proximal and/or distal nerve stump, such as to inform the clinician of the appropriately sized device 100 to deploy. Sizing tool 250 can be constructed and arranged to measure the distance between the proximal and distal never stumps, such as to inform the clinician of the appropriate placement of fixation elements 150.

In some embodiments, tool 200 comprises one, two, or more functional elements, functional element 299 shown. Functional element 299 can comprise one or more components that are integral to deployment tool 210 and/or sizing tool 250. Functional element 299 can comprise a visibility enhancing element configured to improve the ability of the clinician to visualize the field of view, working area, and/or device 100. Functional element 299 can comprise a visibility enhancing element selected from the group consisting of: a high contrast background (e.g. blue, yellow, green, and the like) for the working area; an active lighting component (e.g. bright field, polarized, IR, and/or monochromatic lighting component, such as a fiber optic illuminator); magnification lenses (e.g. lenses with a magnification of 2×, 4×, 10×, and so on), such as lenses with or without a camera sensor; one or more nozzles to deliver a flow of air to clear the field of view and/or working area; one or more nozzles to provide a vacuum to clear the field of view and/or working area; one or more nozzles to provide a stream of saline to rinse and keep hydrated the field of view and/or working area; a downstream structure (e.g. dam) to collect liquids and/or other materials shedding from the field of view and/or working area; and combinations of these. Functional element 299 can comprise a tension feedback element configured to inform the clinician via visual, tactile, and/or other feedback mechanisms when a desired and/or excessive tension is achieved while reapproximating the nerve endings. In some embodiments, if excessive tension is sensed, an interpositional element can be deployed between the proximal and distal nerve stumps. Functional element 299 can comprise a component configured to provide visibility, tension feedback, or both. For example, functional element 299 can provide visual magnification (e.g. under polarized light illumination) to the clinician to visualize the Bands of Fontana, the appearance of which can provide an assessment of the suitable level of stretch for the nerve structure. As another example, functional element 299 can comprise a component configured to allow the clinician to visualize the level of superficial blood perfusion for the nerve, which can indicate when excessive stretch is applied to the nerve (e.g. when blanching starts to occur).

Referring specifically to FIG. 1A, a side-sectional view of a nerve transection is illustrated. As shown, the transected nerve comprises a proximal nerve stump and a distal nerve stump. Device 100 can be configured to provide longitudinal stability (reference I shown), alignment stability (reference II shown), torsional stability (reference III shown), and/or additional features (references IV, V shown) to the transected nerve.

Referring specifically to FIG. 1B, a side-sectional view of a nerve transection is illustrated. As shown, two device 100s can be implanted at each of the proximal and distal stump interfaces. Each device 100 can be configured to provide longitudinal stability (reference I shown), alignment stability (reference II shown), torsional stability (reference III shown), and/or additional features (references IV, V shown) to both the proximal and distal coaptation sites. In some embodiments, a nerve autograft, allograft, and/or conduit is used to bridge a nerve gap injury derived from a traumatic or iatrogenic injury.

Referring specifically to FIG. 1C, a side-sectional view of a nerve amputation and capping device/graft application is illustrated. As shown, one device 100 can be implanted between the amputated nerve stump and a nerve capping device/graft. Device 100 can be configured to provide longitudinal stability (reference I shown), alignment stability (reference II shown), torsional stability (reference III shown), and/or additional features (references IV, V shown) to the implantation site.

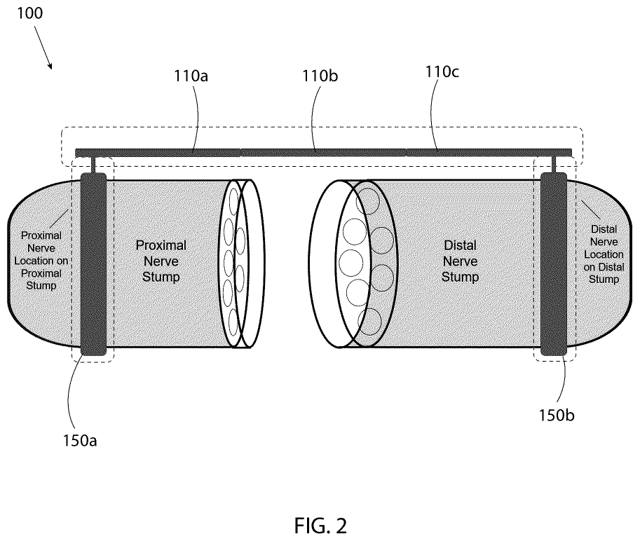

Referring now to FIG. 2, an anatomical side view of an embodiment of a neurorrhaphy device is illustrated, consistent with the present inventive concepts. Device 100, shown at least partially deployed at a nerve transection site in FIG. 2, can comprise two or more longitudinal elements 110, such as 110a-c shown, and two or more fixation elements 150, such as 150a,b shown. Fixation element 150a can be applied to (e.g. fixed to) the proximal nerve stump and fixation element 150b can be applied to the distal nerve stump, with longitudinal elements 110a-c extending therebetween. Longitudinal elements 110a-c can be coupled (e.g. mechanically coupled) to fixation elements 150a,b to stabilize the nerve injury.

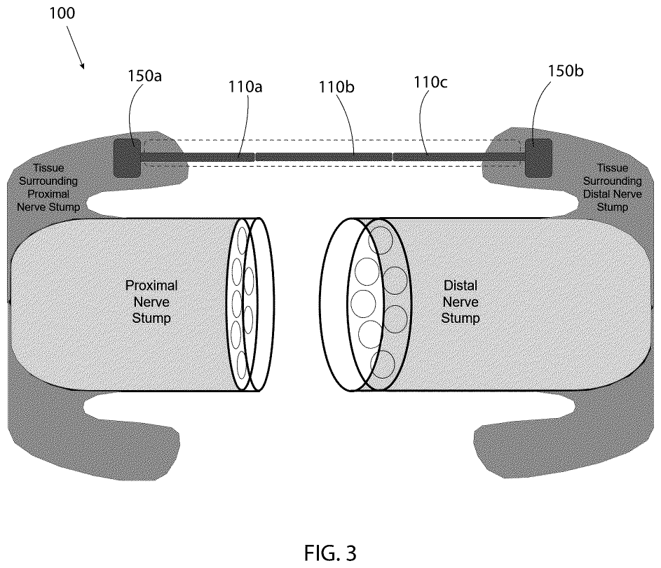

Referring now to FIG. 3, an anatomical side view of an embodiment of a neurorrhaphy device is illustrated, consistent with the present inventive concepts. Device 100, shown at least partially deployed at a nerve transection site in FIG. 3, can comprise two or more longitudinal elements 110, such as 110a-c shown, and two or more fixation elements 150, such as 150a,b shown. Fixation element 150a can be applied to tissue proximate the proximal nerve stump and fixation element 150b can be applied to tissue proximate the distal nerve stump, with longitudinal elements 110a-c extending therebetween. Longitudinal elements 110a-c can be coupled (e.g. mechanically coupled) to fixation elements 150a,b to stabilize the nerve injury.

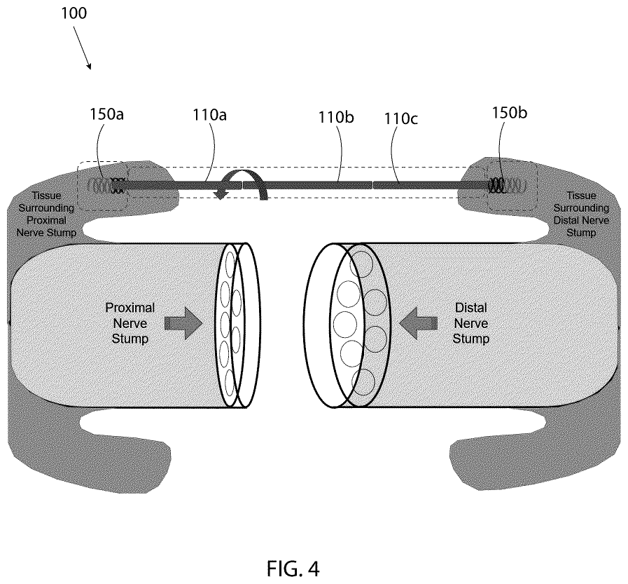

Referring now to FIG. 4, an anatomical side view of an embodiment of a neurorrhaphy device is illustrated, consistent with the present inventive concepts. Device 100, shown at least partially deployed at a nerve transection site in FIG. 4, can comprise two or more longitudinal elements 110, such as 110a-c shown, and two or more fixation elements 150, such as 150a,b shown. Fixation element 150a can be applied to tissue proximate the proximal nerve stump and fixation element 150b can be applied to tissue proximate the distal nerve stump, with longitudinal elements 110a-c extending therebetween. Longitudinal elements 110a-c can be coupled (e.g. mechanically coupled) to fixation elements 150a,b to stabilize the nerve injury.

As shown, fixation elements 150a,b can comprise a helical coil connected to longitudinal elements 110a,c, respectively. Rotation of device 100 (e.g. via a longitudinal element 110) about its longitudinal axis can cause fixation elements 150a,b to progressively penetrate into tissue of the respective nerve stump, thereby increasing the anchoring depth of fixation elements 150a,b into the tissue.

In some embodiments, oppositely-positioned helical couplers have opposite pitch orientation (e.g. left coupler: right-handed pitch; right coupler: left-handed pitch). Clockwise rotation of device 100 (e.g. via a longitudinal element 110) about its longitudinal axis causes fixation elements 150a,b to penetrate the two opposite tissue locations, thereby creating a progressive approximation of (e.g. due to progressive tension applied to) the two tissue locations and reducing tension on the nerve stumps.

Fixation elements 150a,b can comprise one, two, or more materials of selected based on the type and/or geometry of tissue surrounding the proximal and/or distal nerve stumps, and can be further configured to enhance anchoring strength and/or broaden the type of tissue available for use in anchoring.

One, two, or more components of longitudinal elements 110a-c and fixation elements 150a,b can comprise a metal, such as NiTi or similar alloys, stainless steel 316, stainless steel 304, platinum, or degradable magnesium alloys.

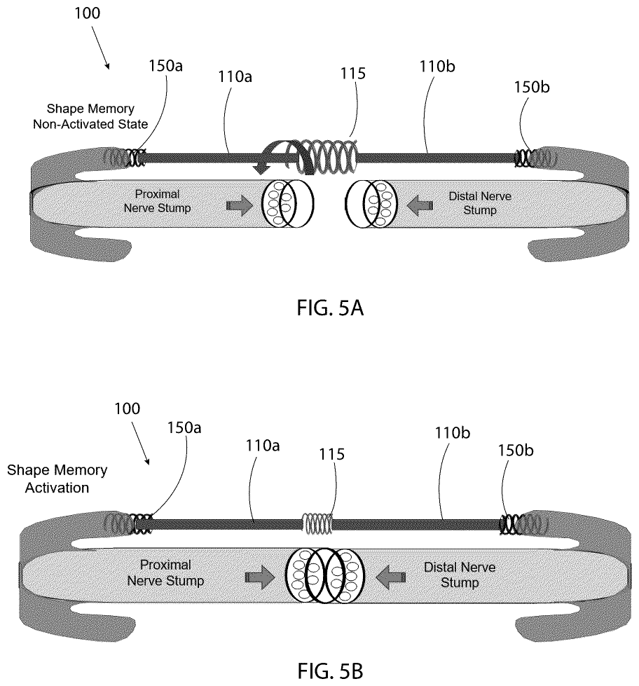

Referring now to FIGS. 5A and 5B, anatomical side views of an embodiment of a neurorrhaphy device is illustrated, consistent with the present inventive concepts. Device 100 can, shown at least partially deployed at a nerve transection site in FIGS. 5A,B, comprise two or more longitudinal elements 110, such as 110a,b shown, and two or more fixation elements 150, such as 150a,b shown. Device 100 can comprise tension adjusting element 115 coupled (e.g. mechanically coupled) between longitudinal elements 110a,b, as shown. Fixation element 150a can be applied to tissue proximate the proximal nerve stump and fixation element 150b can be applied to tissue proximate the distal nerve stump, with longitudinal elements 110a,b and tension adjusting element 115 extending therebetween. Longitudinal elements 110a,b can be coupled (e.g. mechanically coupled) to fixation elements 150a,b to stabilize the nerve injury.

In some embodiments, tension adjusting element 115 comprises a shape memory material (e.g. shape memory, super elastic NiTi) that, upon activation (e.g. electrical, thermal, and the like), is configured to reduce in length thus causing an approximation of the nerve stumps, thereby creating a reduction of tension in the nerve stumps, and coaptation of the nerve stumps (as shown in FIG. 5B).

The cumulative and/or discrete length of longitudinal elements 110a,b can be adjusted to a size needed to approximate and/or align the proximal and distal nerve stumps. In some embodiments, the cumulative and/or discrete length of longitudinal elements 110a,b can be readjusted after implantation into the patient, such as when the initial length was improperly estimated prior to implantation.

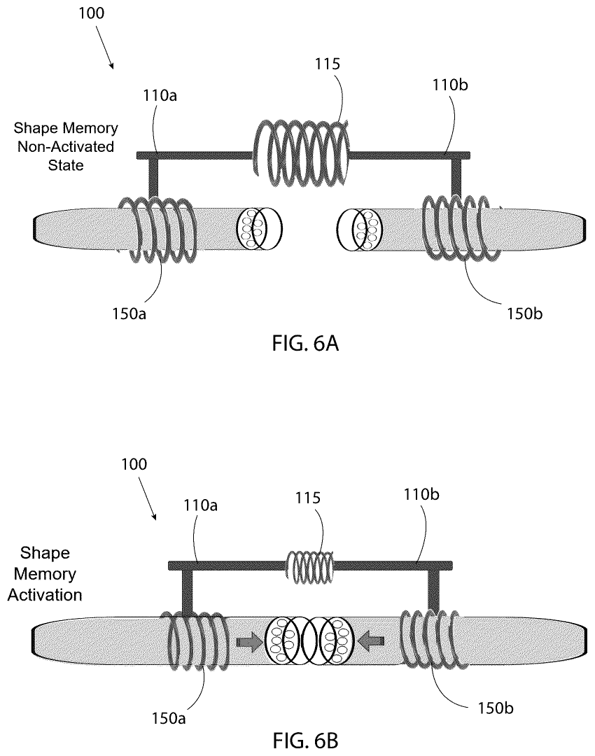

Referring now to FIGS. 6A and 6B, anatomical side views of an embodiment of a neurorrhaphy device is illustrated, consistent with the present inventive concepts. Device 100, shown at least partially deployed at a nerve transection site in FIGS. 6A,B, can comprise two or more longitudinal elements 110, such as 110a,b shown, and two or more fixation elements 150, such as 150a,b shown. Device 100 can comprise tension adjusting element 115 coupled (e.g. mechanically coupled) between longitudinal elements 110a,b, as shown. Fixation element 150a can be applied to the proximal nerve stump and fixation element 150b can be applied to the distal nerve stump, with longitudinal elements 110a,b and tension adjusting element 115 extending therebetween. Longitudinal elements 110a,b can be coupled (e.g. mechanically coupled) to fixation elements 150a,b to stabilize the nerve injury.

As shown, fixation elements 150a,b can each comprise a helical coil which can be positioned concentrically about the proximal and distal nerve stumps, respectively. Fixation elements 150a,b can comprise a textured surface and/or other surface modification or treatment. Fixation elements 150a,b can comprise a shape memory material that, upon activation (e.g. electrical, thermal, and the like) is configured to cause an element 150 to reduce in length and/or diameter causing an approximation of the nerve stumps, thereby creating a reduction of tension in the nerve stumps, and coaptation of the nerve stumps. In some embodiments, fixation elements 150a,b comprise a metal (e.g. nitinol) and/or a plastic comprising an internal textured surface, such that the textured frictional surfaces, in combination with sufficient applied forces, can create frictional engagement with the surrounded tissue to create one or more anchoring points.

Tension adjusting element 115 can comprise a shape memory material that, upon activation (e.g. electrical, thermal, and the like), is configured to reduce in length causing an approximation of the nerve stumps, thereby creating a reduction of tension in the nerve stumps, and coaptation of the nerve stumps (as shown in FIG. 6B). As shown, tension adjusting element 115 can comprise a helical coil coupled (e.g. mechanically coupled) to longitudinal elements 110a,b. In some embodiments, tension adjusting element 115 comprises a shaped memory metal (e.g. nitinol) and/or a shaped memory polymer.

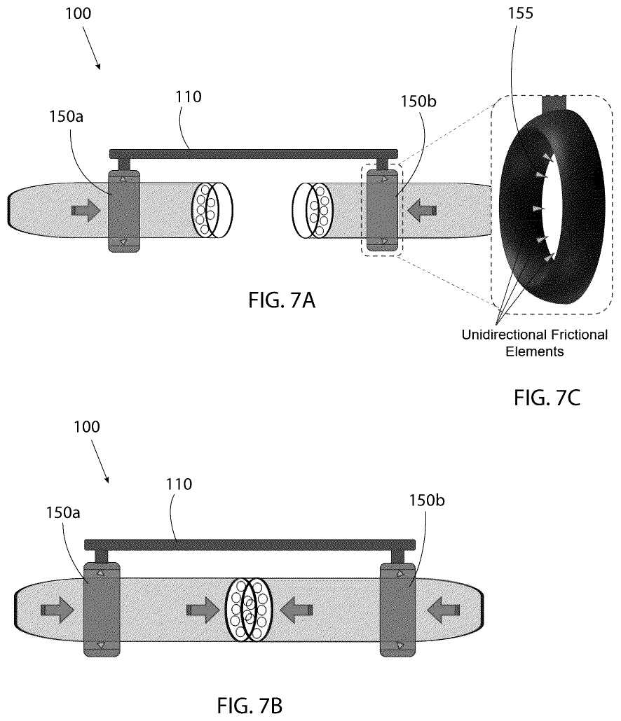

Referring now to FIGS. 7A and 7B, anatomical side views of an embodiment of a neurorrhaphy device is illustrated, consistent with the present inventive concepts. Referring additionally to FIG. 7C, a perspective view of an anchoring component is illustrated, also consistent with the present inventive concepts. Device 100, shown at least partially deployed at a nerve transection site in FIGS. 7A,B, can comprise at least one longitudinal element 110 and two or more fixation elements 150, such as 150a,b shown. Fixation element 150a can be applied to the proximal nerve stump and fixation element 150b can be applied to the distal nerve stump, with longitudinal elements 110 extending therebetween. Longitudinal element 110 can be coupled (e.g. mechanically coupled) to fixation elements 150a,b to stabilize the nerve injury.

As shown, fixation elements 150a,b can comprise cuffs constructed and arranged to slidingly receive at least a portion of the respective nerve stump. In some embodiments, fixation elements 150a,b comprise cuffs constructed as complete loops configured to sliding received at least a portion of the respective nerve stump. In some embodiments, fixation elements 150a,b comprise cuffs constructed as flat sheets configured to fold around the respective nerve stump (e.g. to form a complete loop). Fixation elements 150a,b can each comprise one, two, or more anchoring elements 155 configured to attach fixation element 150 to the nerve stump. Anchoring element 155 can comprise barbs constructed and arranged to penetrate the nerve stump surface (e.g. nerve epineurium). Anchoring element 155 can comprise one or more barbs configured to resist relative rotation between fixation element 150 and the respective nerve stump. In some embodiments, anchoring element 155 comprises bidirectional barbs, such that bidirectional longitudinal friction is created once the nerve stump is inserted into fixation element 150. In some embodiments, anchoring element 155 comprises unidirectional barbs, such that unidirectional longitudinal friction is created once the nerve stump is inserted into fixation element 150 (as shown in FIG. 7C).

In some embodiments, fixation elements 150a,b comprise cuffs constructed of biologically derived materials, such as human amniotic membrane, blood vessels, umbilical cord vessels, and nerve tissue. In some embodiments, fixation elements 150a,b comprise cuffs comprising synthetically derived materials such as silicone, polyester or polyurethane plastics, and polytetrafluorethylene.

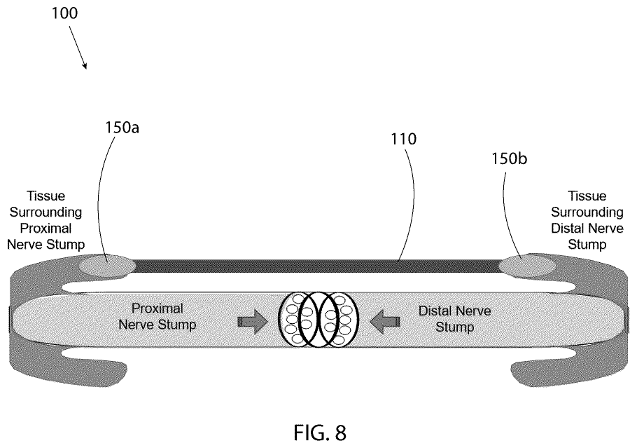

Referring now to FIG. 8, an anatomical side view of an embodiment of a neurorrhaphy device is illustrated, consistent with the present inventive concepts. Device 100, shown at least partially deployed at a nerve transection site in FIG. 8, can comprise longitudinal element 110 and two or more fixation elements 150, such as 150a,b shown. Fixation element 150a can be applied to tissue proximate the proximal nerve stump and fixation element 150b can be applied to tissue proximate the distal nerve stump, with longitudinal element 110 extending therebetween. Longitudinal element 110 can be coupled (e.g. mechanically coupled) to fixation elements 150a,b to stabilize the nerve injury and/or create longitudinal support to reapproximate the free nerve endings.

Fixation elements 150a,b can comprise one, two, or more adhesive elements configured to adhere to tissue proximate the nerve stumps. The adhesive elements can comprise: a biological-based adhesive, such as Fibrin glue, animal or fish-based gelatin, mussel adhesive, mucoadhesive, polysaccharide-based adhesive or gums (e.g. cellulose ethers, chitosan, xanthan gum), and/or hydrogel (e.g. polyethylene glycol-based hydrogels); a chemical-based adhesive, such as a carboxyvinyl copolymer, and polyvinyl alcohol-, cyanoacrylate-, polyacrylic acid-, and polyurethane-based glue; and combinations of these.

In some embodiments, fixation elements 150a,b comprise a polymeric film pad (e.g. a plasma treated pad) to create electrical adhesive interaction with the tissue. In some embodiments, fixation elements 150a,b comprise an adhesive comprising a biologically derived material and is configured to be photochemically bonded to tissue surrounding the nerve stumps.

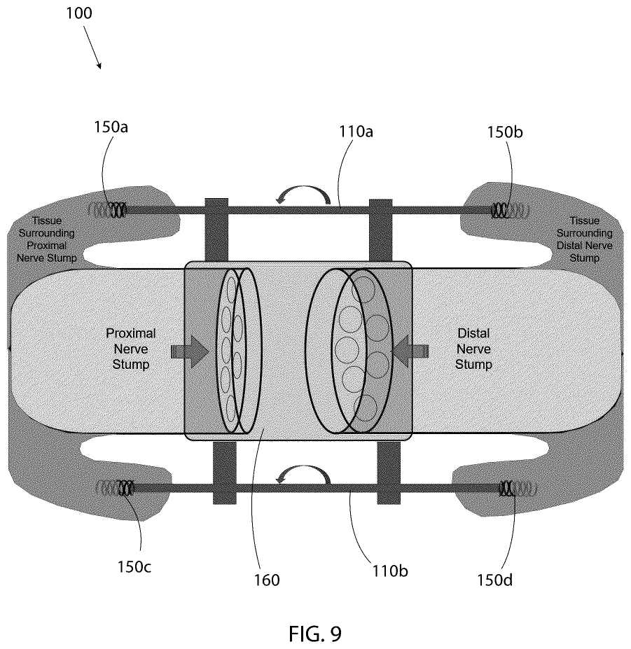

Referring now to FIG. 9, an anatomical side view of an embodiment of a neurorrhaphy device is illustrated, consistent with the present inventive concepts. Device 100, shown at least partially deployed at a nerve transection site in FIG. 9, can comprise two or more longitudinal elements 110, such as 110a,b shown, and two or more fixation elements 150, such as 150a-d shown. Fixation element 150a can be applied to tissue proximate the proximal nerve stump and fixation element 150b can be applied to tissue proximate the distal nerve stump, with longitudinal elements 110a extending therebetween. Fixation element 150c can be applied to tissue proximate the proximal nerve stump and fixation element 150d can be applied to tissue proximate the distal nerve stump, with longitudinal elements 110b extending therebetween.

Device 100 can comprise aligning element 160 configured to surround at least a portion of the proximal and distal nerve stumps, where the internal diameter of aligning element 160 is similar to the external diameter of the nerve stumps. Aligning element 160 can be coupled (e.g. mechanically coupled) to longitudinal elements 110a,b, such as coupled to one, two, or more portions of each longitudinal element.

As shown, fixation elements 150a-d can comprise a helical coil connected to longitudinal elements 110a,b. Rotation of device 100 (e.g. via a longitudinal element 110) about its longitudinal axis can cause fixation elements 150a-d to progressively penetrate into tissue of the respective nerve stump, thereby increasing the anchoring depth between fixation elements 150a-d into the tissue. In some embodiments, oppositely-positioned helical couplers have opposite pitch orientation (e.g. left coupler: right-handed pitch; right coupler: left-handed pitch). Clockwise rotation of device 100 (e.g. via a longitudinal element 110) about its longitudinal axis causes fixation elements 150a-d to penetrate the two opposite tissue locations, thereby creating a progressive approximation of (e.g. due to progressive tension applied to) the two tissue locations and reducing tension on the nerve stumps. Rotation of device 100 (e.g. via a longitudinal element 110) can further cause aligning element 160 to translate relative to longitudinal elements 110a,b, thereby adjusting the positioning of aligning element 160.

In some embodiments, rotation of at least one longitudinal element 110a,b causes aligning element 160 to translate relative to longitudinal elements 110a,b, thereby adjusting the positioning of aligning element 160 about the nerve stumps. In some embodiments, aligning element 160 comprises a soft porous degradable fabric (e.g. a biodegradable fabric).

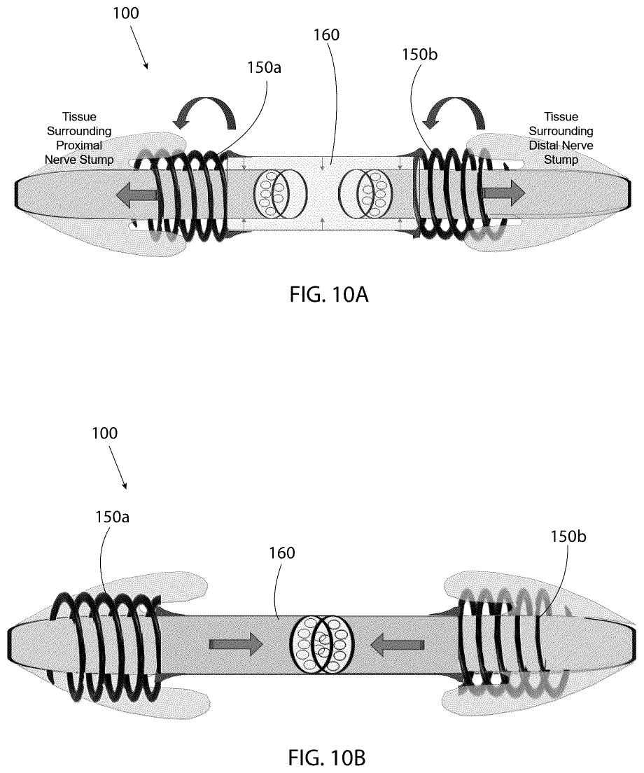

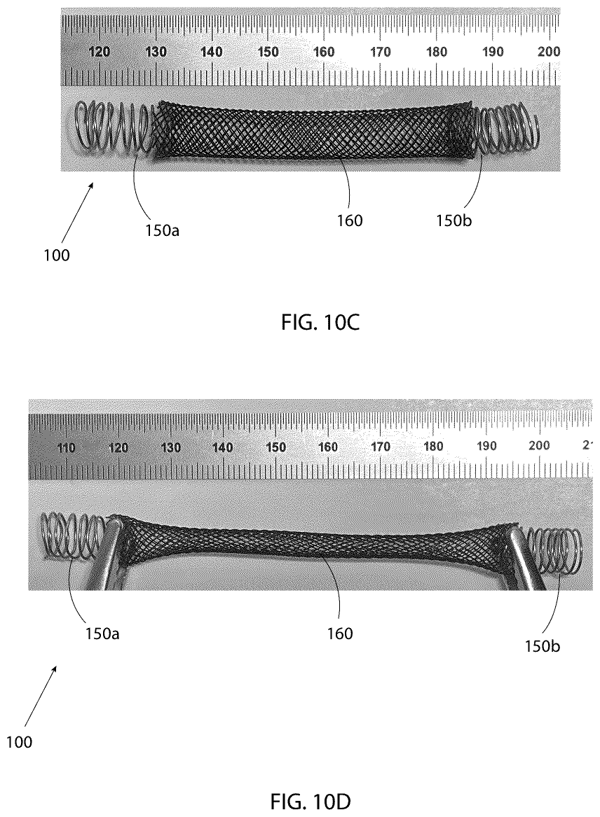

Referring now to FIGS. 10A and 10B, anatomical side views of an embodiment of a neurorrhaphy device is illustrated, consistent with the present inventive concepts. Device 100, shown at least partially deployed at a nerve transection site in FIGS. 10A,B, can comprise two or more fixation elements 150, such as 150a,b shown, and an aligning element 160. Fixation element 150a can be applied to tissue proximate the proximal nerve stump and fixation element 150b can be applied to tissue proximate the distal nerve stump, with aligning element 160 extending therebetween. Aligning element 160 can be configured to surround at least a portion of the proximal and distal nerve stumps, such that the internal diameter of aligning element 160 is similar to the external diameter of the nerve stumps. As described herein, aligning element 160 can comprise a mesh cuff. Aligning element 160 can be coupled (e.g. mechanically coupled) to fixation elements 150a,b. In some embodiments, aligning element 160 comprises a cylindrical mesh configured to reduce its internal diameter when longitudinal tension and stretch is applied via fixation elements 150a,b. Aligning element 160 can comprise a braided, knitted, or woven cylindrical mesh comprising threads constructed from one, two or more synthetic nondegradable and/or degradable polymers, such as nylon, polypropylene, PTFE, polyesters, polyglactin, polyurethanes, polyamides, and/or other polymers. Aligning element 160 can also comprise a braided, knitted, and/or woven cylindrical mesh comprising threads constructed from one, two, or more biologically-derived materials, such as collagen, elastin, proteoglycans, polycarbonates, glycosaminoglycans, and/or extracellular matrix-derived materials. Aligning element 160 comprising one, two, or more braided, knitted, and/or woven materials can comprise a material selected from the group consisting of: biological degradable sutures, such as plain gut or chromic gut; biological non-degradable sutures, such as silk; synthetic degradable sutures, such PLLA, PLA, PGA, PCA, PLLA-PGA; non degradable sutures, such as nylon, polypropylene, PTFE, PET, and PETG; and combinations of these.

In some embodiments, the material used for braiding, knitting, and/or weaving comprise monofilament threads obtained from extracellular matrices, such as amnion, small intestine submucosa, urinary bladder mucosa, and others. These monofilament threads can comprise extracellular matrix obtained from a series of mechanical and/or chemical treatments configured to clean and decellularize tissue (e.g. recently harvested tissue). The extracellular matrix obtained can then be cut to comprise elongate elements that can be twisted, stretched, and dried to the desired length.

In some embodiments, monofilament threads used for braiding, knitting, and/or weaving aligning element 160 can be post-processed with mechanical, chemical, and/or physical treatments to modify (e.g. improve) their properties. For example, mechanical conditioning (e.g. cyclic stretching) can be configured to remove residual stresses in the structure and/or make the structure more supple. As another example, chemical processes can be configured to increase the durability, flexibility, strength, and/or other properties of the threads. As another example, chemical functionalization can be configured to increase adhesive or antibacterial properties of the thread surface. As another example, treatment with heat or plasma can be configured to increase the mechanical or adhesive properties of the threads.

In some embodiments, monofilaments obtained from extracellular matrix can be configured to alter one, two, or more of its properties upon rehydration, thereby resulting in a structural and/or chemical change in the material. For example, twisted filaments can be configured to untwist and swell upon absorbing water, which can cause the structure to become adhesive due to water intermolecular bonds between the hydrated filaments and between the hydrated filaments and tissue. As another example, when twisted monofilaments are braided, knitted, and/or woven, a swelling of the monofilaments can result in a mechanical interference and/or seizing of the structure. Hydration mediated seizing can be used to lock the relative position of the filaments among each other and secure tissue within the braided, knitted, and/or woven aligning element 160. As another example, when twisted monofilaments are rehydrated, the swelling and partial untwisting can increase the adhesive properties of threads within the surrounding tissues.

As shown, fixation elements 150a,b can each comprise a helical coil which can be positioned concentrically about the proximal and distal nerve stumps, respectively. Rotation of device 100 (e.g. via a fixation element 150) about its longitudinal axis can cause fixation elements 150a,b to progressively penetrate into tissue of the respective nerve stump, thereby increasing the anchoring depth of fixation elements 150a,b into the tissue. In some embodiments, oppositely-positioned helical couplers have opposite pitch orientation (e.g. left coupler: right-handed pitch; right coupler: left-handed pitch). Clockwise rotation of device 100 (e.g. via a fixation element 150) about its longitudinal axis causes fixation elements 150a,b to penetrate the two opposite tissue locations, thereby creating a progressive approximation of (e.g. due to progressive tension applied to) the two tissue locations and reducing tension on the nerve stumps (as shown in FIG. 10B). In some embodiments, rotation of device 100 (e.g. via a fixation element 150) causes aligning element 160 to translate, thereby adjusting the positioning of aligning element 160 about the nerve stumps.

Referring additionally to FIGS. 10C and 10D, top-view photographs of a prototypical neurorrhaphy device comprising a woven element in a relaxed and stretched state are illustrated, respectively.

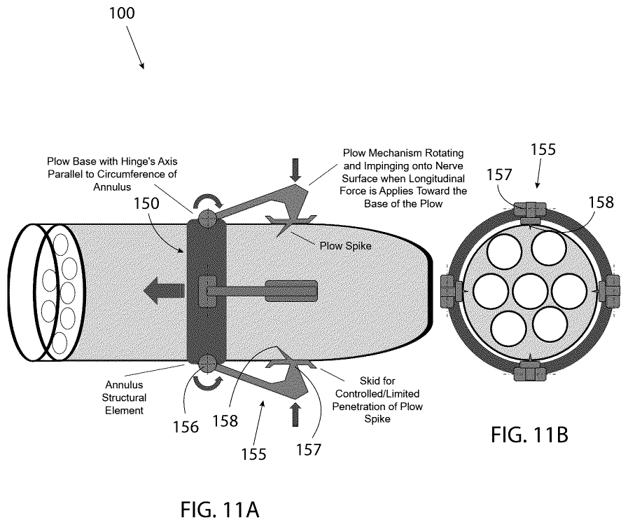

Referring now to FIGS. 11A and 11B, anatomical side views of an embodiment of a nerve fixation element including anchoring elements is illustrated, consistent with the present inventive concepts. Fixation elements 150, shown at least partially deployed at a nerve transection site in FIGS. 11A,B, can be constructed and arranged to create one or more anchoring points in a location and manner that is non-traumatic for the nerve's internal structure (e.g. fascicles). As shown, fixation elements 150 can comprise a cuff constructed and arranged to slidingly receive at least a portion of the nerve stump.

Fixation element 150 can comprise one, two, or more anchoring elements 155 configured to attach fixation element 150 to the nerve stump. Anchoring element 155 can comprise an anchor (e.g. a plow tether as shown) that is constructed and arranged to penetrate the nerve stump surface (e.g. nerve epineurium). For example, each plow tether can comprise a base 156, skid 157, and spike 158. Base 156 can be coupled (e.g. mechanically coupled) to fixation element 150 via a hinge, such that anchoring element 155 can rotate relative to fixation element 150. Skid 157 can be constructed and arranged to control and/or limit the penetration of spike 158 into the nerve stump surface. When longitudinal force is applied to anchoring element 155, base 156 rotates to bring spike 158 into contact with the nerve stump (as shown in FIG. 11B).

Fixation element 150 and/or anchoring element 155 can comprise one, two, or more metals, such as stainless steel, Nitinol, platinum, or magnesium. Fixation element 150 and/or anchoring element 155 can comprise one, two, or more synthetic degradable and/or nondegradable polymers, such as PTFE, polyesters, polyurethanes, polyamides, and/or other polymers.

Fixation element 150 can comprise one, two, or more adhesive elements configured to adhere to tissue proximate the nerve stump. The adhesive elements can comprise: a biological-based adhesive, such as Fibrin glue, animal or fish-based gelatin, mussel adhesive, mucoadhesive, polysaccharide-based adhesive or gums (e.g. cellulose ethers, chitosan, xanthan gum), and/or hydrogel (e.g. polyethylene glycol-based hydrogels); a chemical-based adhesive, such as a carboxyvinyl copolymer, and polyvinyl alcohol-, cyanoacrylate-, polyacrylic acid-, and polyurethane-based glue; and combinations of these.

At least one surface of fixation element 150 can comprise a texture configured to improve its frictional properties. At least one surface of fixation element 150 can comprise a texture configured to stimulate underlying nerve tissue via chemical treatments, abrasive texturization, treatments to create a sequential directional partial cutting texture (e.g. fish scale texture), and/or other material removal strategies.

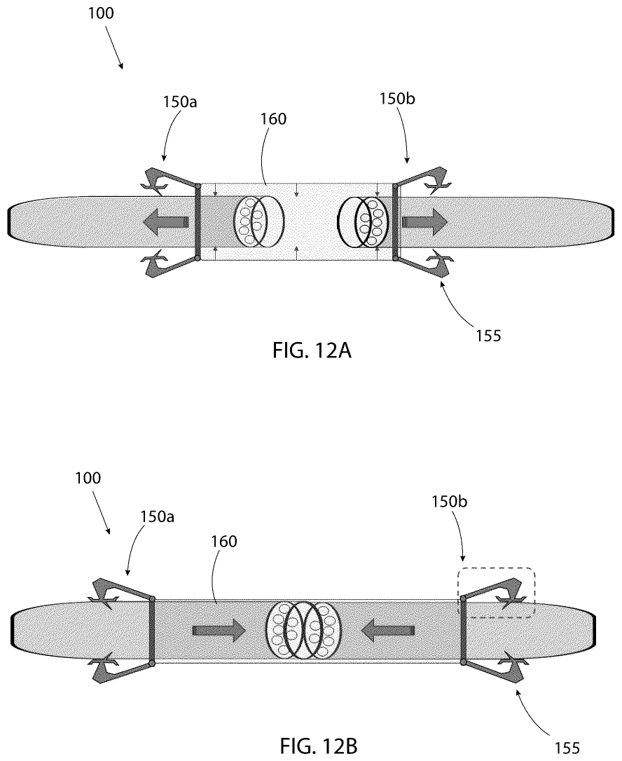

Referring now to FIGS. 12A and 12B, anatomical side views of an embodiment of a neurorrhaphy device is illustrated, consistent with the present inventive concepts. Device 100, shown at least partially deployed at a nerve transection site in FIGS. 12A,B, can comprise two or more fixation elements 150, such as 150a,b shown, and an aligning element 160. Fixation element 150a can be applied to the proximal nerve stump and fixation element 150b can be applied to the distal nerve stump, with aligning element 160 extending therebetween. Aligning element 160 can be configured to surround at least a portion of the proximal and distal nerve stumps, such that the internal diameter of aligning element 160 is similar to the external diameter of the nerve stumps. Aligning element 160 can comprise a mesh cuff constructed and arranged similar to that as described hereinabove in reference to FIGS. 10A-D. Aligning element 160 can be coupled (e.g. mechanically coupled) to fixation elements 150a,b.

Fixation elements 150a,b can each comprise one, two, or more anchoring elements 155 configured to attach fixation element 150 to the nerve stump. Anchoring elements 155 can comprise one, two, or more physical tethers (e.g. non-traumatic tethers), such as helical coils anchored to surrounding tissue, unidirectional frictional elements, and/or barbs. Anchoring elements 155 can comprise one, two, or more adhesive elements (e.g. non-traumatic tethers), such as adhesive strips comprising biologically and/or chemically derived glues, pads with an adhesive, and/or similar elements placed circumferentially onto the peripheral circular edges of the aligning element 160. As shown, anchoring element 155 can comprise a plow tether or other anchor constructed and arranged to penetrate the nerve stump surface (e.g. nerve epineurium). Anchoring element 155 can be constructed and arranged as described hereinabove in reference to FIGS. 11A,B.

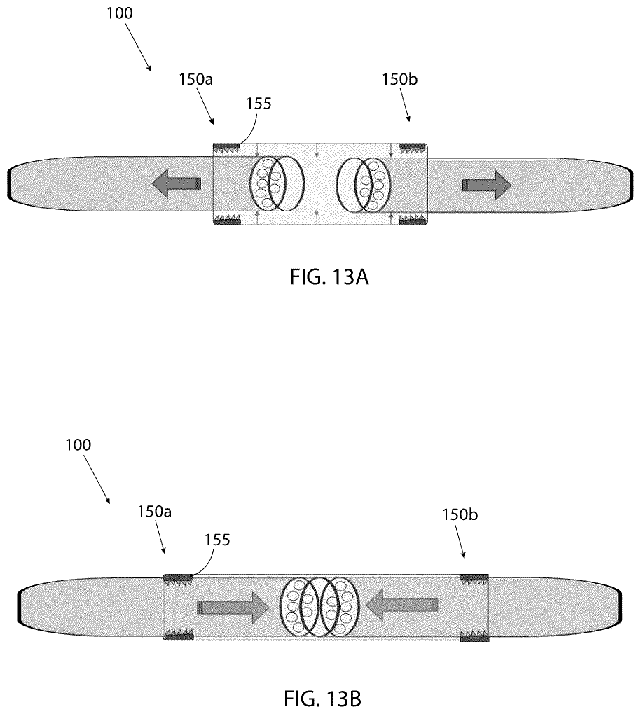

Referring now to FIGS. 13A and 13B, anatomical side views of an embodiment of a neurorrhaphy device is illustrated, consistent with the present inventive concepts. Device 100, shown at least partially deployed at a nerve transection site in FIGS. 13A,B, can comprise two or more fixation elements 150, such as 150a,b shown, and an aligning element 160. Fixation element 150a can be applied to the proximal nerve stump and fixation element 150b can be applied to the distal nerve stump, with aligning element 160 extending therebetween. In some embodiments, fixation elements 150a,b extend throughout the entire length of device 100. Aligning element 160 can be configured to surround at least a portion of the proximal and distal nerve stumps, such that the internal diameter of aligning element 160 is similar to the external diameter of the nerve stumps. Aligning element 160 can comprise a mesh cuff constructed and arranged similar to that as described hereinabove in reference to FIGS. 10A-D. Aligning element 160 can be coupled (e.g. mechanically coupled) to fixation elements 150a,b.

Fixation elements 150a,b can each comprise one, two, or more anchoring elements 155 configured to attach fixation element 150 to the nerve stump. As shown, anchoring element 155 can comprise barbed hooks, scales, or other geometry intended to generate friction configured to penetrate, indent, or otherwise interfere with the nerve stump surface (e.g. nerve epineurium). In some embodiments, anchoring element 155 comprises unidirectional barbed hooks, scales, or other geometry intended to generate friction that are constructed and arranged continuously or intermittently around the internal circumference of aligning element 160. Anchoring element 155 can comprise a metal selected from the group consisting of: stainless steel, such as 316L; cobalt-chromium alloy; Nitinol; platinum; tantalum alloy; and combinations of these.

In some embodiments, anchoring element 155 comprises an extension of the braided, knitted, or woven mesh fibers comprising aligning element 160 that may be reinforced with chemical, physical, or mechanical treatments. In some embodiments, anchoring element 155 comprises a separate element constructed and arranged as needed about the peripheral boundaries of aligning element 160 and the proximal and/or distal nerve stumps to hold aligning element 160 in place.

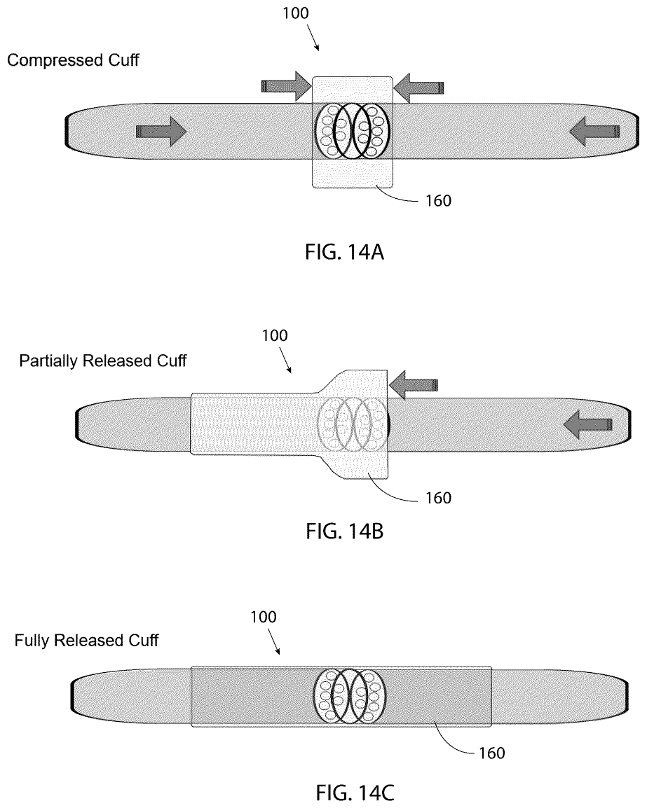

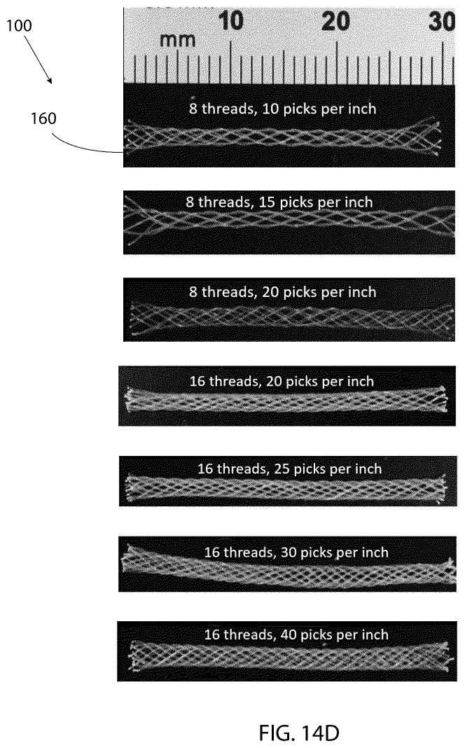

Referring now to FIGS. 14A-C, anatomical side views of an embodiment of a neurorrhaphy device is illustrated, consistent with the present inventive concepts. Device 100, shown at least partially deployed at a nerve transection site in FIGS. 14A-C, can comprise an aligning element 160. As shown, a first end of aligning element 160 can slidingly receive the proximal nerve stump and a second end of aligning element 160 can slidingly receive the distal nerve stump. Aligning element 160 can comprise a mesh cuff constructed and arranged similar to that as described hereinabove in reference to FIGS. 10A-D.

Aligning element 160 can comprise a braided, knitted, or woven mesh cuff constructed with threads (e.g. textured threads) configured to be longitudinally compressed prior to insertion of the nerve stumps, thereby causing an increase in diameter and reduction in length of aligning element 160. While in the compressed state, aligning element 160 can slidingly receive at least a portion of the nerve stumps held in a reapproximated position. The compression of aligning element 160 can then be slowly released, causing a reduction in diameter and an increase in length, which entraps the nerve stumps therein (as shown in FIGS. 14A,B).

Aligning element 160 can be sized and constructed such as to enable aligning element 160 to surround a portion of each nerve stump, entrap and hold the nerve stumps, and resist the specific longitudinal and torsional forces that tend to pull and misalign the nerve stumps of different sizes. For example, when fully compressed along its main axis, the inner diameter of the aligning element 160 can be configured to be larger than the specific diameter of the proximal and distal nerve stumps. As another example, upon releasing the aforementioned compression, the length and inner diameter of aligning element 160 can return to a neutral state in which the length of aligning element 160 is sufficient to overlap portions of proximal and distal nerve stumps, and the inner diameter of aligning element 160 is smaller than that of the nerve stumps. This results in a controlled radial compression of the outer surface of the nerve stumps via aligning element 160, which ensures the nerve stump coaptation area has sufficient longitudinal torsional and alignment stability. Additionally, configuration enables a negative feedback loop, in which increasing longitudinal forces pulling the nerve stumps apart (e.g. forces attributed to normal body motion) result in an increase in compressive radial forces around the nerve stumps, thereby preventing translational disengagement of either nerve stump.

In some embodiments, aligning element 160 is actively rehydrated prior to deployment, such as to improve its deployment by providing lubrication between aligning element 160, deployment tool 210, and/or between aligning element 160 and the nerve stumps. In some embodiments, aligning element 160 is passively rehydrated upon deployment in the body, thereby improving the adhesiveness of aligning element 160 to the nerve stumps and/or the responsiveness of the aforementioned negative feedback loop. Rehydration of aligning element 160 can also activate functional element 199 in device 100, such as an activation of a lubrication, adhesive, and/or release of factors aimed at improving nerve healing.

A textured and/or otherwise modified surface of aligning element 160 can be configured to further prevent an unintended translation of the nerve stumps therein, as well as further ensure the coaptation area has sufficient longitudinal torsional, and alignment stability. In some embodiments, an internal surface of aligning element 160 can be texturized via a process selected from the group consisting of: chemical or plasma treatment; mechanical material removal processes, such as sanding, electrosurgical cutting, ultrasound cutting, laser manufacturing techniques; additive and/or texturing processes such as electrospinning, coating, use of adhesives, creation of a textured surface; and combinations of these.

In some embodiments, aligning element 160 comprises one, two, or more synthetically- and/or biologically-derived materials configured to be further secured to the proximal and/or distal nerve stump via an adhesive and/or chemical, frictional, and/or photochemical bonding process.

In some embodiments, aligning element 160 comprises a lubricious external surface configured to enable aligning element 160 to slide uninhibited within the surrounding tissues during the normal movement of the body without generating significant frictional resistance, irritation, inflammation, adhesions, or injury. The lubricious external surface of aligning element 160 can be achieved via a surface treatment process selected from the group consisting of: chemical or plasma treatment; mechanical material removal processes, such as sanding, electrosurgical cutting, ultrasound cutting, laser manufacturing techniques; additive or texturing processes, such as electrospinning, coating, use of lubricants, creation of a textured surface; and combinations of these.

In some embodiments, aligning element 160 comprises a braided configuration constructed from 2-0, 3-0, 4-0, 5-0, and/or 6-0 monofilament plain gut suture, or other suture sizes selected based on the size of the nerves and intended application. Aligning element 160 can comprise a cylindrical braid constructed by braiding 8, 16, 24, 32, and/or 48 suture threads in 1:1, 1:2, 2:2, and/or other number of suture threads and braiding patterns based on the size of nerves and intended application. The cylindrical braid can comprise a braid angle (e.g. half the angle made by crossing filaments in the braid, or the positive angle between the central axis of the braid and the crossing filaments of the braid) comprising an angle of between 5 and 85 degrees, and which defines the braid pick per unit length (such that 1 pick is 1 repeat of a braiding pattern along the central braid axis). Aligning element 160 can be braided over one, two, or more mandrels each comprising a diameter of between 1 mm and 10 mm.

Different combinations of the braiding parameters, as described herein, can result in braid porosities within aligning element 160 of between 10% and 90%. Braid porosity can be configured to affect the transmural visibility of aligning element 160, which can aid the clinician in establishing a desired nerve stump coaptation distance and alignment during deployment. Braid porosity can be configured to affect the degradation rate of aligning element 160 and the exchange of nutrients between the surrounding tissues and the nerve stumps. Braid porosity can be configured to affect the access of cells derived from the surrounding tissue to the coaptation area.

The proximal and/or distal ends of a braided aligning element 160 can be subjected to the tendency to splice. In some embodiments, thread splicing can be prevented at the proximal and/or distal ends of aligning element 160 via: braiding in a closed-loop configuration; applying additional weaved, braided, knitted, and/or glued threads, or bands of material around the circumferential direction; treating one or multiple locations, including a complete circumferential band proximate to the proximal and/or distal ends with heat, compression, adhesives or other chemicals to foster material adhesion; knotting; permanent deformations, such as outward revolving of threads to create a permanent loop; and combinations thereof.

Aligning element 160 can comprise a braided cuff constructed according to one, two, or more parameters specific to nerve size and application. For example, aligning element 160 can comprise a braided configuration comprising a neutral state (e.g. absent an application of compression and/or tensional forces) constructed to comprise an internal diameter of between 70% and 90% of the intended nerve diameter application. These levels of diameter interference are necessary to allow aligning element 160 to support the required longitudinal and torsional forces to maintain the coaptation between the two nerve stumps. The internal diameter of aligning element 160 in a neutral state can be controlled by the size of the braiding mandrel upon which the braiding process is performed. For example, depending on the number of braiding threads and braiding pattern, the braiding mandrel should be sized to be between 10% and 50% larger than the intended internal diameter of aligning element 160. This oversizing is necessary to account for the reduction in diameter occurring when the braid is removed from the braid mandrel, caused by the intrinsic circumferential stresses generated during the braiding process. The length of aligning element 160 in its neutral state also depends on specific nerve size and application, braid construction parameters, and/or potential internal surface texturization applied to the device. In some embodiments, aligning element 160 can comprise a length constructed to enable an overlap of aligning element 160 over each nerve stump to be between 2 and 10 times the diameter of the nerve stump. These levels of overlap enable aligning element 160 to support the required longitudinal and torsional forces to maintain the coaptation between the two nerve stumps. For example, a nerve stump of 3 mm in diameter requires aligning element 160 to comprise a length of between 12 mm and 60 mm. If a defined distance between nerve stumps is desired, such distance should be added to the total length of aligning element 160.

Referring specifically to FIG. 14D, aligning element 160 can comprise a braided cuff constructed from 8 or 16 wires comprising 5-0 monofilament plain gut suture. In some embodiments, aligning element 160 comprises a braided cuff constructed using a 1:1 or 2:2 braiding pattern, braided over a mandrel of approximately 2.5 mm in diameter, with a length comprised between 20 mm and 30 mm. The resulting braid porosity of aligning element 160 comprises a porosity of between 20% and 60%, thereby corresponding to a picks per inch of 10, 15, 20, 25, 30, and/or 40, as shown, and a range of braid angles of between 25% and 35%.

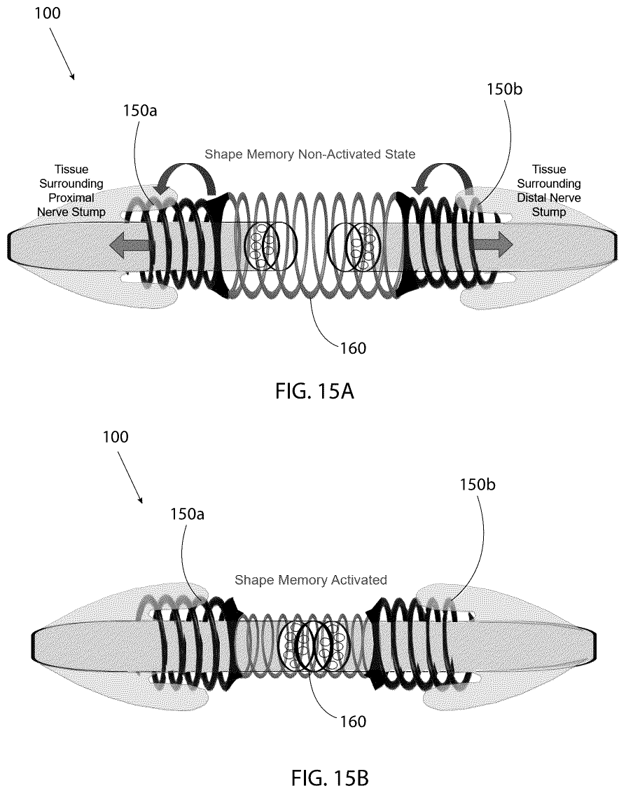

Referring now to FIGS. 15A and 15B, anatomical side views of an embodiment of a neurorrhaphy device is illustrated, consistent with the present inventive concepts. Device 100, shown at least partially deployed at a nerve transection site in FIGS. 15A,B, can comprise two or more fixation elements 150, such as 150a,b shown, and an aligning element 160. Fixation element 150a can be applied to the proximal nerve stump and fixation element 150b can be applied to the distal nerve stump, with aligning element 160 extending therebetween. Aligning element 160 can be configured to surround at least a portion of the proximal and distal nerve stumps, such that the internal diameter of aligning element 160 is similar to the external diameter of the nerve stumps. Aligning element 160 can be coupled (e.g. mechanically coupled) to fixation elements 150a,b.

As shown, fixation elements 150a,b can each comprise a helical coil which can be positioned concentrically about the proximal and distal nerve stumps, respectively. Fixation elements 150a,b can comprise a shape memory material that, upon activation (e.g. electrical, thermal, and the like) is configured to reduce in length and/or diameter causing an approximation of the nerve stumps, thereby creating a reduction of tension in the nerve stumps, and coaptation of the nerve stumps. In some embodiments, fixation elements 150a,b comprise super-elastic metal (e.g. super-elastic nitinol).

Aligning element 160 can comprise a helical coil which can be positioned concentrically about a portion of the proximal and distal nerve stumps. Aligning element 160 can comprise a shape memory material that, upon activation (e.g. electrical, thermal, and the like) is configured to reduce in length and/or diameter causing an approximation of the nerve stumps, thereby creating a reduction of tension of the nerve stumps, and coaptation of the nerve stumps (as shown in FIG. 15B). In some embodiments, aligning element 160 comprises super-elastic metal (e.g. super-elastic nitinol).

Rotation of device 100 (e.g. via a fixation element 150) about its longitudinal axis can cause fixation elements 150a,b to progressively penetrate into tissue of the respective nerve stump, thereby increasing the anchoring depth of fixation elements 150a,b into the tissue. In some embodiments, oppositely-positioned helical couplers have opposite pitch orientation (e.g. left coupler: right-handed pitch; right coupler: left-handed pitch). Clockwise rotation of device 100 (e.g. via a fixation element 150) about its longitudinal axis causes fixation elements 150a,b to penetrate the two opposite tissue locations, thereby creating a progressive approximation of (e.g. due to progressive tension applied to) the two tissue locations and reducing tension on the nerve stumps.

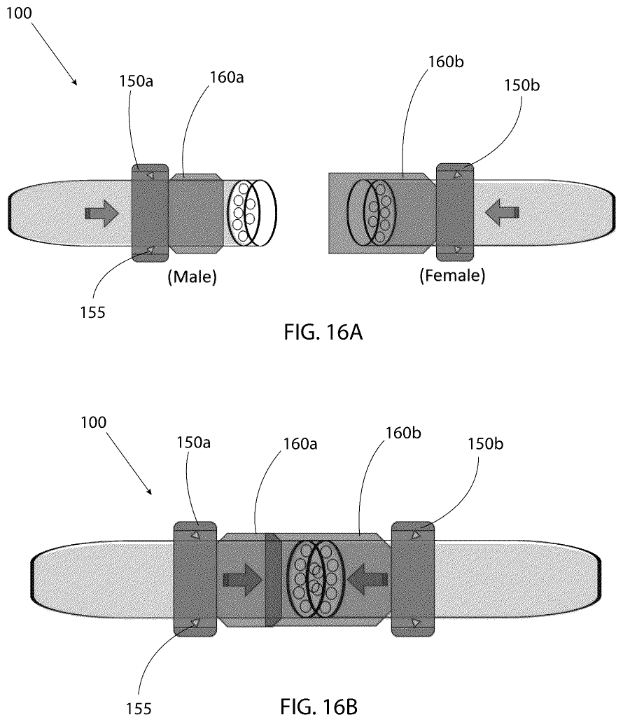

Referring now to FIGS. 16A and 16B, anatomical side views of an embodiment of a neurorrhaphy device is illustrated, consistent with the present inventive concepts. Device 100, shown at least partially deployed at a nerve transection site in FIGS. 16A,B, can comprise two or more fixation elements 150, such as 150a,b shown. Device 100 can comprise two or more fixation elements 150, such as 150a,b shown, and two or more aligning elements 160, such as 160a,b shown. Fixation element 150a can be applied to the proximal nerve stump and fixation element 150b can be applied to the distal nerve stump, with aligning elements 160a,b extending therebetween. Aligning elements 160a,b can be configured to surround at least a portion of the proximal and distal nerve stumps, respectively, such that the internal diameter of aligning elements 160a,b are similar to the external diameter of the nerve stumps. Aligning elements 160a,b can be coupled (e.g. mechanically coupled) to fixation elements 150a,b, respectively. In some embodiments, device 100 comprises one or more elements (e.g. spacer 170) positioned between aligning elements 160a,b and configured to provide a desired spacing between the nerve stumps.

Fixation elements 150a,b can each comprise one, two, or more anchoring elements 155 configured to attach fixation element 150 to the nerve stump. As shown, anchoring element 155 can comprise barbs constructed and arranged to penetrate the nerve stump surface (e.g. nerve epineurium). Anchoring element 155 can comprise barbs configured to resist relative rotation between fixation element 150 and the respective nerve stump. In some embodiments, anchoring element 155 comprises bidirectional barbs, such that bidirectional longitudinal friction is created once the nerve stump is inserted into fixation element 150. In some embodiments, anchoring element 155 comprises unidirectional barbs, such that unidirectional longitudinal friction is created once the nerve stump is inserted into fixation element 150. Fixation element 150 can comprise one, two, or more materials selected from the group consisting of: metals, such as biologically compatible metals; degradable polymers; non-degradable polymers; biologically derived materials; and combinations of these.

Aligning elements 160a,b can be constructed and arranged to engage (e.g. connect) with the other (as shown in FIG. 16B), such as via a snap locking feature, twisting via thread, magnetic forces, mechanical interference, crimping, adhesive forces and/or elements, photochemical bonding, and the like. Aligning elements 160a,b can be configured to resist relative rotation between aligning element 160 and the respective nerve stump. In some embodiments, aligning elements 160a,b comprise an internal surface texture and/or other surface treatment configured to create a bidirectional longitudinal friction with the exterior surface of the nerve stump. In some embodiments, aligning elements 160a,b comprise an internal surface texture and/or other surface treatment configured to create a unidirectional longitudinal friction with the exterior surface of the nerve stump. Aligning elements 160a,b can comprise one or more durable (e.g. metals, alloys, polymers, and the like) and/or degradable (e.g. biodegradable metals, alloys, polymer, biological materials, and the like) materials. Aligning elements 160a,b can be constructed and arranged to provide selective permeability to the nerve coaptation site.

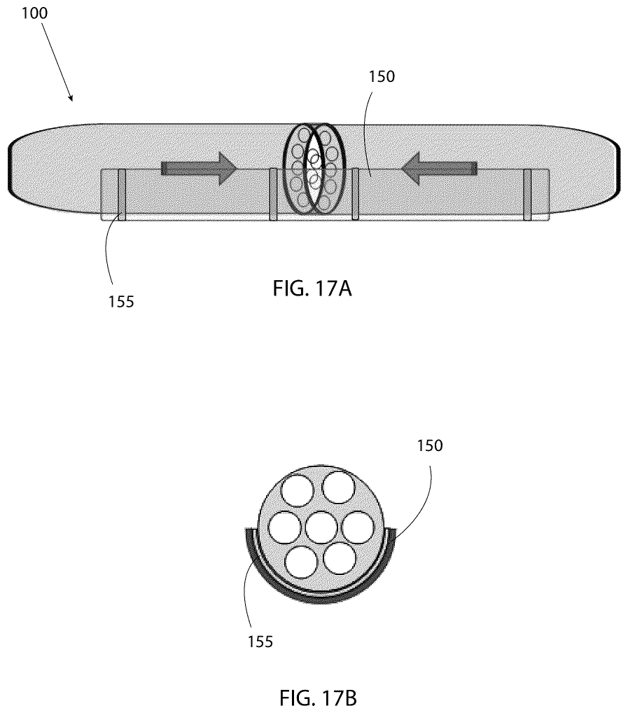

Referring now to FIGS. 17A and 17B, an anatomical side view and end view, respectively, of an embodiment of a neurorrhaphy device is illustrated, consistent with the present inventive concepts. Device 100, as shown at least partially deployed at a nerve transection, can comprise at least one fixation element 150 comprising a semi-cuff constructed and arranged to slidingly receive at least a portion of each nerve stump. As shown, a first portion of fixation element 150 can slidingly receive the proximal nerve stump and a second portion of fixation element 150 can slidingly receive the distal nerve stump. Fixation element 150 can comprise woven threads fabricated in a semi-cylindrical structure (e.g. a degradable structure).

Fixation element 150 can comprise one, two, or more anchoring elements 155 configured to attach fixation element 150 to the nerve stumps. As shown, anchoring element 155 can comprise an adhesive configured to adhere to the nerve stump.

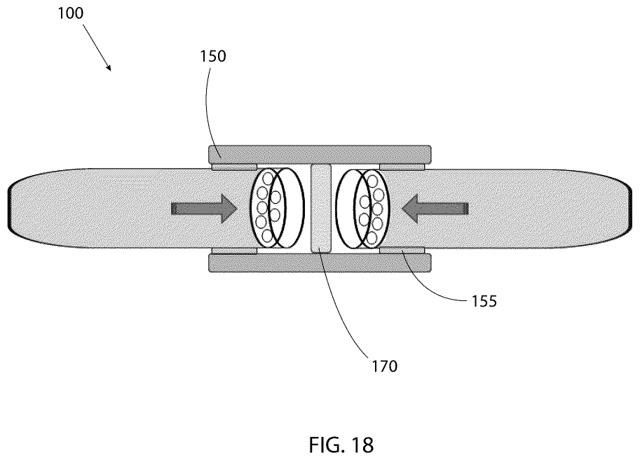

Referring now to FIG. 18, an anatomical side view of an embodiment of a neurorrhaphy device is illustrated, consistent with the present inventive concepts. Device 100, shown at least partially deployed at a nerve transection site in FIG. 18, can comprise at least one fixation element 150 comprising a cuff constructed and arranged to slidingly receive at least a portion of each nerve stump. As shown, a first end of fixation element 150 can slidingly receive the proximal nerve stump and a second end of fixation element 150 can slidingly receive the distal nerve stump. In some embodiments, fixation element 150 comprises a porous, degradable material (e.g. a biodegradable material).

Fixation element 150 can comprise one, two, or more anchoring elements 155 configured to attach fixation element 150 to the nerve stumps. As shown, anchoring element 155 can comprise adhesive bands configured to adhere to tissue proximate the nerve stumps. In some embodiments, anchoring element 155 is lubricious (e.g. prior to a hydration step) and is configured to become adhesive once the nerve stumps are inserted into fixation element 150 (e.g. and fixation element 150 is subsequently hydrated, such as with saline). Fixation element 150 can comprise one, two, or more adhesive materials selected from the group consisting of: water soluble polymers derived from cellulose ethers; polyvinyl acetates; carbomers; polysaccharide gums; starches; gelatin; carboxyvinyl copolymers; polyacrylic acids; polyvinyl alcohols; alginate; casein; pullulan; and combinations of these.

Device 100 can comprise a spacer 170 positioned within fixation element 150. As shown, spacer 170 can comprise a degradable (e.g. biodegradable) porous material comprising a morphology configured to support neurite penetration. Spacer 170 can comprise a biological material, such as one, two, or more extracellular matrix components. Spacer 170 can comprise a synthetic material.

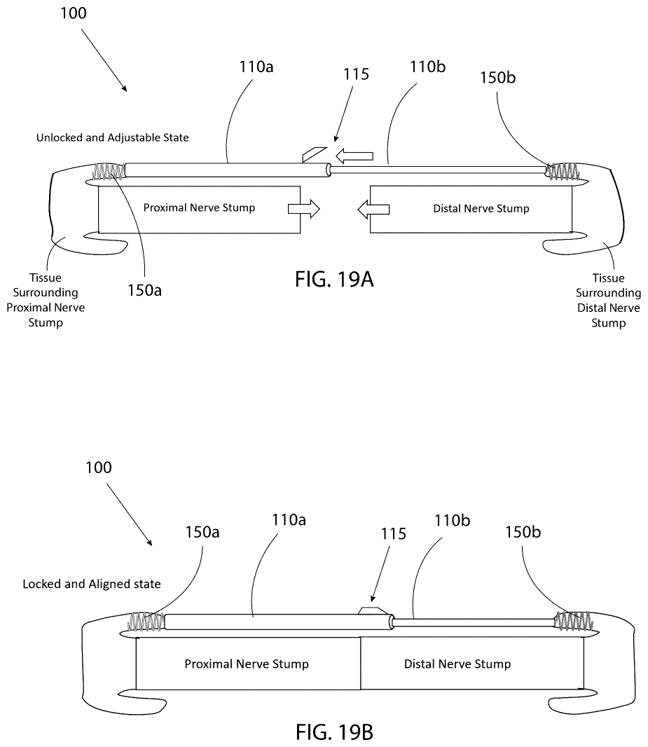

Referring now to FIGS. 19A and 19B, anatomical side views of an embodiment of a neurorrhaphy device is illustrated, consistent with the present inventive concepts. Device 100 can, shown at least partially deployed at a nerve transection site in FIGS. 19A,B, comprise two or more longitudinal elements 110, such as 110a,b shown, and two or more fixation elements 150, such as 150a,b shown. Device 100 can comprise tension adjusting element 115 coupled (e.g. mechanically coupled) between longitudinal elements 110a,b, as shown. Fixation element 150a can be applied to tissue proximate the proximal nerve stump and fixation element 150b can be applied to tissue proximate the distal nerve stump, with longitudinal elements 110a,b extending therebetween. Longitudinal elements 110a,b can be coupled (e.g. mechanically coupled) to fixation elements 150a,b to stabilize the nerve injury.

The cumulative and/or discrete length of longitudinal elements 110a,b can be adjusted to a size needed to approximate and/or align the proximal and distal nerve stumps. In some embodiments, the cumulative and/or discrete length of longitudinal elements 110a,b can be readjusted after implantation into the patient, such as when the initial length was improperly estimated prior to implantation. In some embodiments, the cumulative length of longitudinal elements 110a,b is adjustable via a locking, telescopic pole system. Longitudinal element 110a can be constructed and arranged to slidingly receive at least a portion of longitudinal element 110b

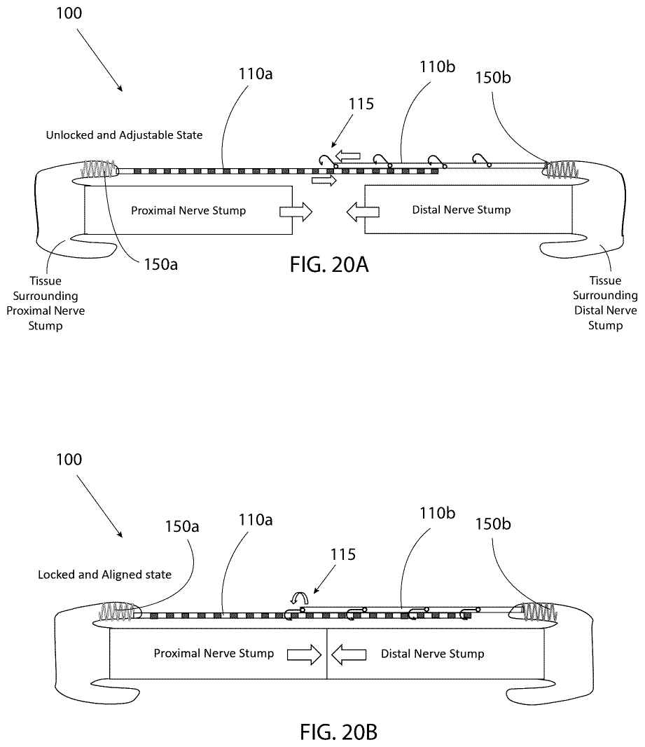

Referring now to FIGS. 20A and 20B, anatomical side views of an embodiment of a neurorrhaphy device is illustrated, consistent with the present inventive concepts. Device 100 can, shown at least partially deployed at a nerve transection site in FIGS. 20A,B, comprise two or more longitudinal elements 110, such as 110a,b shown, and two or more fixation elements 150, such as 150a,b shown. Fixation element 150a can be applied to tissue proximate the proximal nerve stump and fixation element 150b can be applied to tissue proximate the distal nerve stump, with longitudinal elements 110a,b extending therebetween. Longitudinal elements 110a,b can be coupled to fixation elements 150a,b to stabilize the nerve injury.

The cumulative and/or discrete length of longitudinal elements 110a,b can be adjusted to a size needed to approximate and/or align the proximal and distal nerve stumps. In some embodiments, the cumulative and/or discrete length of longitudinal elements 110a,b can be readjusted after implantation into the patient, such as when the initial length was improperly estimated prior to implantation. In some embodiments, the cumulative length of longitudinal elements 110a,b is adjustable via a guided hook and ladder system. Longitudinal element 110a can comprise one, two, or more openings and longitudinal element 110b can comprise one, two, or more hooks. At least one hook of longitudinal element 110b can be configured to engage at least one opening of longitudinal element 110a

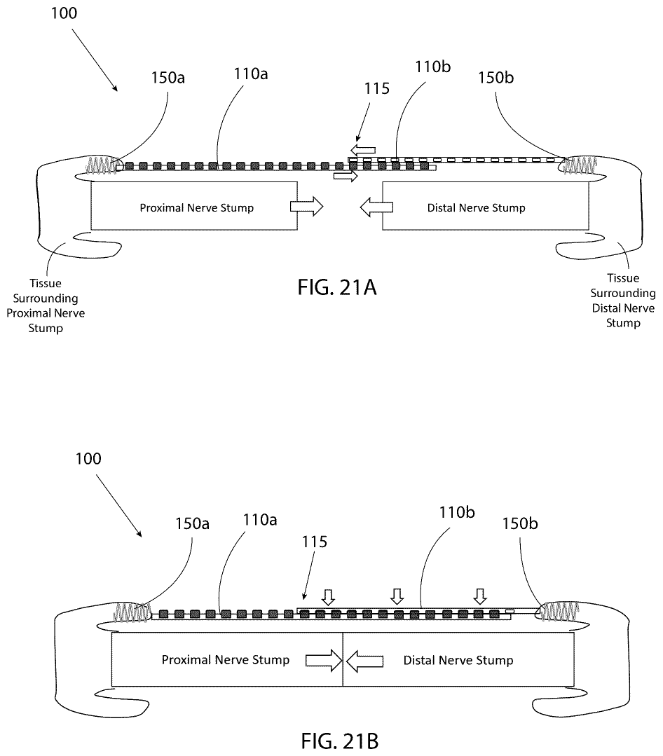

Referring now to FIGS. 21A and 21B, anatomical side views of an embodiment of a neurorrhaphy device is illustrated, consistent with the present inventive concepts. Device 100 can, shown at least partially deployed at a nerve transection site in FIGS. 21A,B, comprise two or more longitudinal elements 110, such as 110a,b shown, and two or more fixation elements 150, such as 150a,b shown. Device 100 can comprise tension adjusting element 115 coupled (e.g. mechanically coupled) between longitudinal elements 110a,b, as shown. Fixation element 150a can be applied to tissue proximate the proximal nerve stump and fixation element 150b can be applied to tissue proximate the distal nerve stump, with longitudinal elements 110a,b extending therebetween. Longitudinal elements 110a,b can be coupled (e.g. mechanically coupled) to fixation elements 150a,b to stabilize the nerve injury.

The cumulative and/or discrete length of longitudinal elements 110a,b can be adjusted to a size needed to approximate and/or align the proximal and distal nerve stumps. In some embodiments, the cumulative and/or discrete length of longitudinal elements 110a,b can be readjusted after implantation into the patient, such as when the initial length was improperly estimated prior to implantation. In some embodiments, the cumulative length of longitudinal elements 110a,b is adjustable via a peg and hole system. Longitudinal element 110a can comprise one, two, or more pegs and longitudinal element 110b can comprise one, two, or more holes. At least one peg of longitudinal element 110a can be configured to engage at least one hole of longitudinal element 110b.

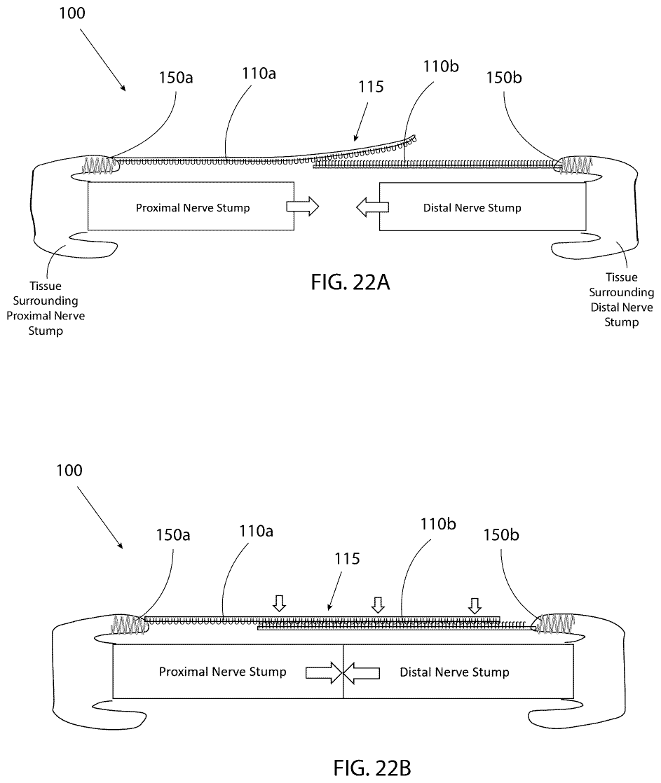

Referring now to FIGS. 22A and 22B, anatomical side views of an embodiment of a neurorrhaphy device is illustrated, consistent with the present inventive concepts. Device 100 can, shown at least partially deployed at a nerve transection site in FIGS. 22A,B, comprise two or more longitudinal elements 110, such as 110a,b shown, and two or more fixation elements 150, such as 150a,b shown. Device 100 can comprise tension adjusting element 115 coupled (e.g. mechanically coupled) between longitudinal elements 110a,b, as shown. Fixation element 150a can be applied to tissue proximate the proximal nerve stump and fixation element 150b can be applied to tissue proximate the distal nerve stump, with longitudinal elements 110a,b extending therebetween. Longitudinal elements 110a,b can be coupled (e.g. mechanically coupled) to fixation elements 150a,b to stabilize the nerve injury.

The cumulative and/or discrete length of longitudinal elements 110a,b can be adjusted to a size needed to approximate and/or align the proximal and distal nerve stumps. In some embodiments, the cumulative and/or discrete length of longitudinal elements 110a,b can be readjusted after implantation into the patient, such as when the initial length was improperly estimated prior to implantation. In some embodiments, the cumulative length of longitudinal elements 110a,b is adjustable via a hook and loop system. Longitudinal element 110a can comprise one, two, or more loops and longitudinal element 110b can comprise one, two, or more hooks. At least one hook of longitudinal element 110b can be configured to engage at least one loop of longitudinal element 110a. The proximal and distal nerve stumps can be reapproximated and secured with fascicular alignment, such that engagement of the loops of longitudinal element 100a and the hooks of longitudinal element 110b are configured to allow for rotational freedom during reapproximation.

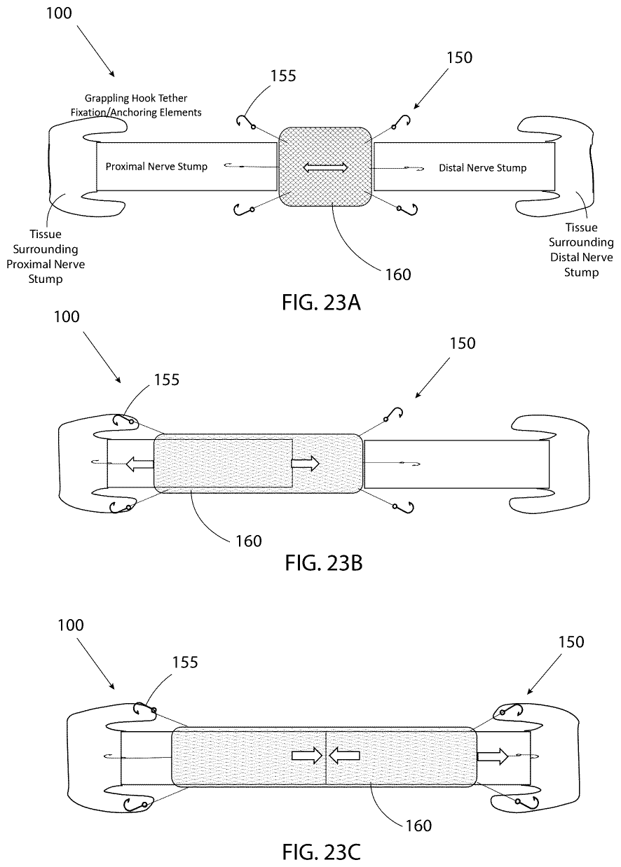

Referring now to FIGS. 23A-C, anatomical side views of an embodiment of a neurorrhaphy device is illustrated, consistent with the present inventive concepts. Device 100, shown at least partially deployed at a nerve transection site in FIGS. 23A-C, can comprise two or more fixation elements 150, such as 150a,b shown, and an aligning element 160. Fixation element 150a can be applied to the proximal nerve stump and fixation element 150b can be applied to the distal nerve stump, with aligning element 160 extending therebetween. Aligning element 160 can be configured to surround at least a portion of the proximal and distal nerve stumps, such that the internal diameter of aligning element 160 is similar to the external diameter of the nerve stumps. Aligning element 160 can be coupled (e.g. mechanically coupled) to fixation elements 150a,b.

Fixation elements 150a,b can comprise one or more components comprising a degradable and/or non-degradable material. In some embodiments, fixation element 150 comprises a mono- or multifilament suture. Fixation elements 150a,b can further comprise one, two, or more anchoring elements 155 configured to attach fixation element 150 to tissue surrounding the nerve stump. As shown, anchoring elements 155 can comprise a hook constructed and arranged to penetrate tissue surrounding the nerve stump. Each anchoring element 155 can comprise a material selected from the group consisting of: stainless steel, such as 316L or 304; metal, such as degradable magnesium alloys; and/or degradable and/or nondegradable plastics. The length of fixation elements 150a,b can be adjusted (e.g. independently adjusted) to achieve a desired nerve reapproximation, such that the proximal and distal nerve stumps can be desirably aligned and reapproximated.

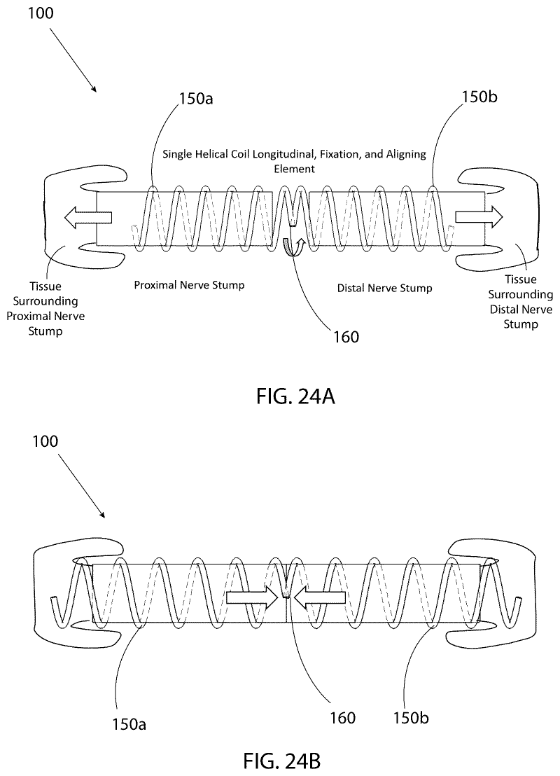

Referring now to FIGS. 24A and 24B, anatomical side views of an embodiment of a neurorrhaphy device is illustrated, consistent with the present inventive concepts. Device 100, shown at least partially deployed at a nerve transection site in FIGS. 24A,B, can comprise two or more fixation elements 150, such as 150a,b shown, and an aligning element 160. Fixation element 150a can be applied to the proximal nerve stump and fixation element 150b can be applied to the distal nerve stump, with aligning element 160 extending therebetween. Aligning element 160 can be configured to surround at least a portion of the proximal and distal nerve stumps, such that the internal diameter of aligning element 160 is similar to the external diameter of the nerve stumps.

As shown, fixation elements 150a,b can each comprise a helical coil which can be positioned concentrically about the proximal and distal nerve stumps, respectively. Aligning element 160 can comprise a helical coil which can be positioned concentrically about a portion of the proximal and distal nerve stumps. Aligning element 160 can be coupled (e.g. mechanically coupled) and/or integral to fixation elements 150a,b, such that fixation elements 150a,b and aligning element 160 comprise a single helical coil. Fixation elements 150a,b and/or aligning element 160 can each comprise a super-elastic metal (e.g. super-elastic nitinol).

Device 100 can comprise a pitch and/or inner diameter constructed and arranged to vary along its length. For example, the distal ends of fixation elements 150a,b can comprise a conical shape comprising a larger pitch, such that device 100 can be securely anchored to tissue surrounding the nerve stumps while the proximal ends of fixation elements 150a,b and/or aligning element 160 can comprise a smaller pitch around the region of coaptation such that the nerve stumps can be held tightly in their realigned position.

Rotation of device 100 (e.g. via a fixation element 150 and/or aligning element 160) about its longitudinal axis can cause fixation elements 150a,b to progressively penetrate into tissue of the respective nerve stump, thereby increasing the anchoring depth of fixation elements 150a,b into the tissue. In some embodiments, fixation elements 150a,b are constructed and arranged to comprise opposite pitch orientations (e.g. fixation element 150a comprises a left-handed pitch and fixation element 150b comprises a right-handed pitch, or vice versa). Clockwise rotation of device 100 (e.g. via a fixation element 150 and/or aligning element 160) about its longitudinal axis causes fixation elements 150a,b to penetrate the two opposite tissue locations, thereby creating a progressive approximation of (e.g. due to progressive tension applied to) the two tissue locations and reducing tension on the nerve stumps.

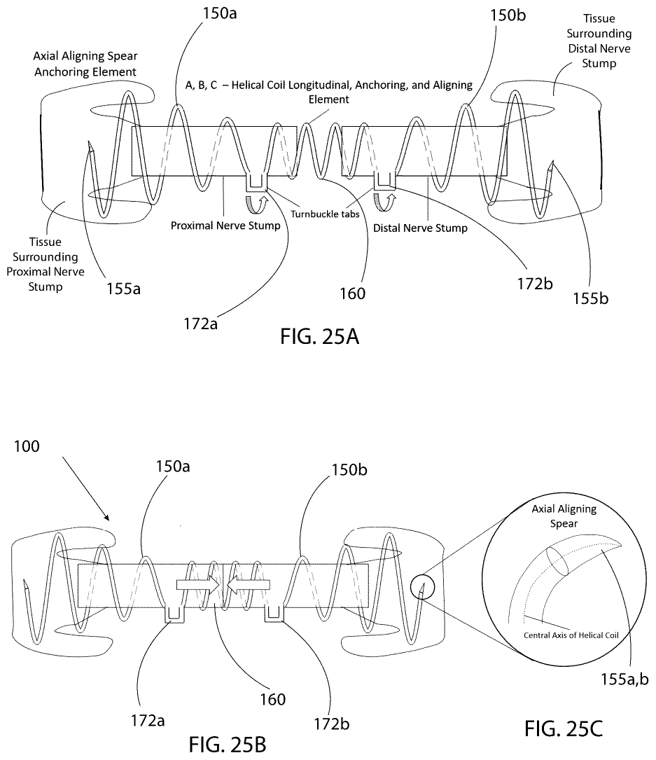

Referring now to FIGS. 25A-C, anatomical side views of an embodiment of a neurorrhaphy device is illustrated, consistent with the present inventive concepts. Device 100, shown at least partially deployed at a nerve transection site in FIGS. 25A-C, can comprise two or more fixation elements 150, such as 150a,b shown, and an aligning element 160. Fixation element 150a can be applied to the proximal nerve stump and fixation element 150b can be applied to the distal nerve stump, with aligning element 160 extending therebetween. Aligning element 160 can be configured to surround at least a portion of the proximal and distal nerve stumps, such that the internal diameter of aligning element 160 is similar to the external diameter of the nerve stumps. Device 100 can further comprise one, two, or more coupling elements 172, such as 172a,b shown, configured to assist in a rotation of fixation elements 150a,b and/or aligning element 160.

Fixation elements 150a,b can each comprise a helical coil which can be positioned concentrically about the proximal and distal nerve stumps, respectively. Fixation elements 150a,b can each further comprise a distal end comprising an anchoring element 155a,b, respectively. As shown in FIG. 25C, anchoring element 155a,b can comprise a spear aligned with the central axis of the respective fixation element 150. In this embodiment, anchoring elements 155a,b are configured to be inserted into tissue and along a path that is concentric with the nerve stump. Aligning element 160 can comprise a helical coil which can be positioned concentrically about a portion of the proximal and distal nerve stumps.

Device 100 can comprise a pitch and/or inner diameter constructed and arranged to vary along its length. For example, the distal ends of fixation elements 150a,b can comprise a conical shape comprising a larger pitch, such that device 100 can be securely anchored to tissue surrounding the nerve stumps while the proximal ends of fixation elements 150a,b and/or aligning element 160 can comprise a smaller pitch around the region of coaptation such that the nerve stumps can be held tightly in their realigned position. In some embodiments, the pitch orientations along the length of device 100 are constructed and arranged to transition from a first pitch orientation (e.g. left-handed pitch) to a second pitch orientation (e.g. right-handed pitch) via a coupling element 172.

Rotation of device 100 (e.g. via at least one of coupling elements 172a,b) about its longitudinal axis can cause fixation elements 150a,b to progressively penetrate (via anchoring elements 155a,b) into tissue, thereby increasing the anchoring depth of fixation elements 150a,b into the tissue. In some embodiments, fixation elements 150a,b are constructed and arranged to comprise opposite pitch orientations (e.g. fixation element 150a comprises a left-handed pitch and fixation element 150b comprises a right-handed pitch, or vice versa). Clockwise rotation of device 100 (e.g. via at least one of coupling elements 172a,b) about its longitudinal axis causes fixation elements 150a,b to penetrate (via anchoring elements 155a,b) the two opposite tissue locations, thereby creating a progressive approximation of (e.g. due to progressive tension applied to) the two tissue locations and reducing tension on the nerve stumps.

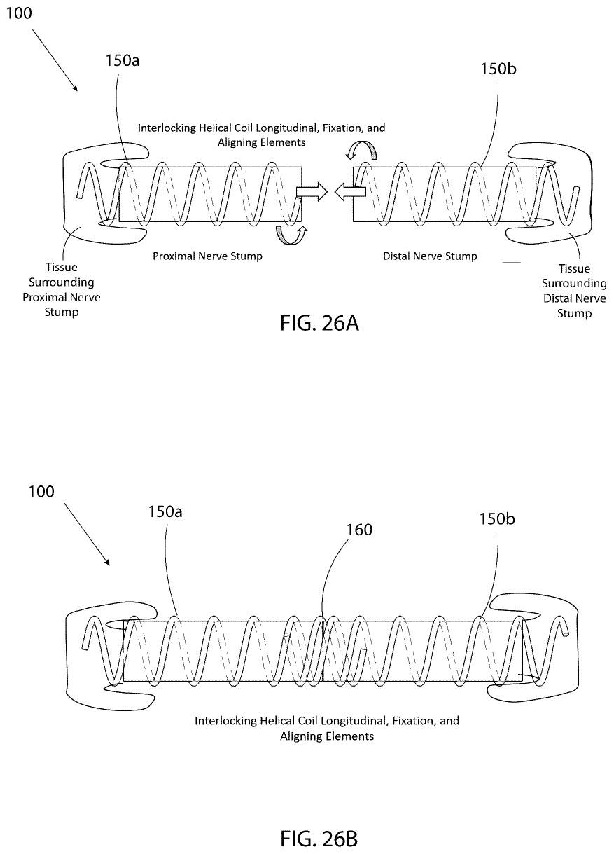

Referring now to FIGS. 26A and 26B, anatomical side views of an embodiment of a neurorrhaphy device is illustrated, consistent with the present inventive concepts. Device 100, shown at least partially deployed at a nerve transection site in FIGS. 26A,B, can comprise two or more fixation elements 150, such as 150a,b shown. Fixation elements 150a,b can each comprise a helical coil which can be positioned concentrically about the proximal and distal nerve stumps, respectively. As described herein, fixation elements 150a,b can be constructed and arranged to interact to form an aligning element 160.

Device 100 can comprise a pitch and/or inner diameter constructed and arranged to vary along its length. For example, the distal ends of fixation elements 150a,b can comprise a conical shape comprising a larger pitch, such that device 100 can be securely anchored to tissue surrounding the nerve stumps while the proximal ends of fixation elements 150a,b and/or aligning element 160 can comprise a smaller pitch around the region of coaptation such that the nerve stumps can be held tightly in their realigned position.

Rotation of fixation elements 150a,b about their longitudinal axis can cause the distal ends of each fixation element to progressively penetrate into tissue of the respective nerve stump, thereby increasing the anchoring depth of fixation elements 150a,b into the tissue. In some embodiments, fixation elements 150a,b are constructed and arranged to comprise opposite pitch orientations (e.g. fixation element 150a comprises a left-handed pitch and fixation element 150 comprises a right-handed pitch, or vice versa). Once the distal ends of fixation elements 150a,b are sufficiently secured within nerve stump tissue, the proximal ends of fixation elements 150a,b can be brought into physical proximity to each other. As shown in FIG. 26B, rotation of the proximal ends of fixation elements 150a,b can be constructed and arranged to interlock the fixation elements to form an aligning element 160. Further rotation of the proximal ends of fixation elements 150a,b can be configured to decrease the distance between the proximal and distal nerve stumps, thereby creating a desired nerve coaptation, such that the proximal and distal nerve stumps can be desirably aligned and reapproximated. Fixation elements 150a,b can comprise an elastic metal, such as Nitinol and/or similar alloy.

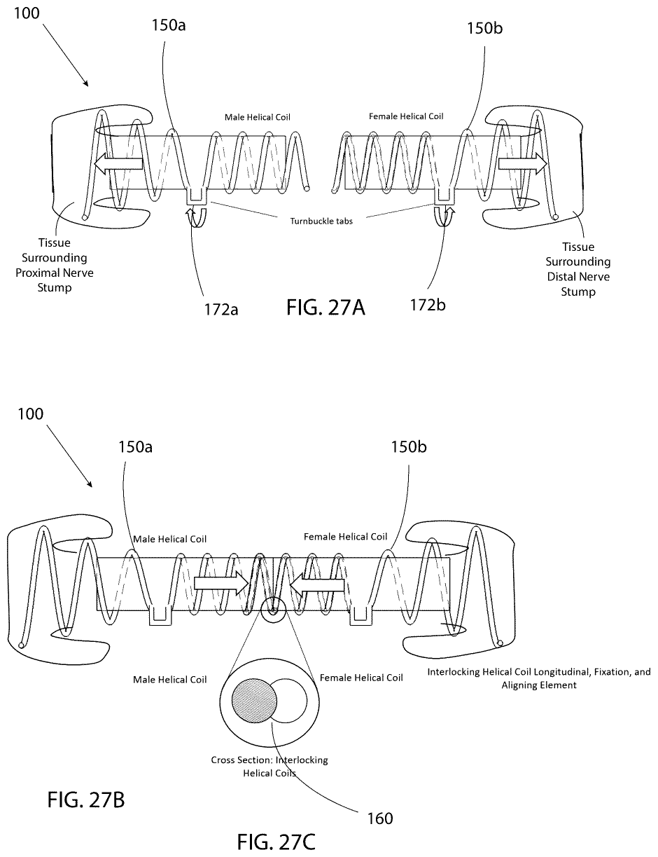

Referring now to FIGS. 27A-C, anatomical side views of an embodiment of a neurorrhaphy device is illustrated, consistent with the present inventive concepts. Device 100, shown at least partially deployed at a nerve transection site in FIGS. 27A,B, can comprise two or more fixation elements 150, such as 150a,b shown. Fixation elements 150a,b can each comprise a helical coil which can be positioned concentrically about the proximal and distal nerve stumps, respectively. As described herein, fixation elements 150a,b can be constructed and arranged to interlock to form an aligning element 160. Device 100 can further comprise one, two, or more coupling elements 172, such as 172a,b shown, configured to assist in a rotation of fixation elements 150a,b and/or aligning element 160.

Fixation element 150a can comprise a helical coil comprising an internal female thread and fixation element 150b can comprise a helical coil comprising an internal male thread, such that the internal female thread of fixation element 150a is configured to engage the internal male thread of fixation element 150b, thereby forming aligning element 160. In an alternate embodiment, fixation element 150a comprises the internal male thread and fixation element 150b comprises the internal female thread.

Device 100 can comprise a pitch and/or inner diameter constructed and arranged to vary along its length. For example, the distal ends of fixation elements 150a,b can comprise a conical shape comprising a larger pitch, such that device 100 can be securely anchored to tissue surrounding the nerve stumps while the proximal ends of fixation elements 150a,b and/or aligning element 160 can comprise a smaller pitch around the region of coaptation such that the nerve stumps can be held tightly in their realigned position.

Rotation of fixation elements 150a,b (e.g. via coupling elements 172a,b) about their longitudinal axis can cause the distal ends of each fixation element to progressively penetrate into tissue of the respective nerve stump, thereby increasing the anchoring depth of fixation elements 150a,b into the tissue. In some embodiments, fixation elements 150a,b are constructed and arranged to comprise opposite pitch orientations (e.g. fixation element 150a comprises a left-handed pitch and fixation element 150b comprises a right-handed pitch, or vice versa). Once the distal ends of fixation elements 150a,b are sufficiently secured within nerve stump tissue, the proximal ends of fixation elements 150a,b can be brought into physical proximity to each other. As shown in FIG. 27C, the internal female thread of fixation element 150a is configured to engage the internal male thread of fixation element 150b, thereby forming aligning element 160. The engagement of fixation elements 150a,b and formation of alignment element 160 can be configured to decrease the distance between the proximal and distal nerve stumps, thereby creating a desired nerve coaptation, such that the proximal and distal nerve stumps can be desirably aligned and reapproximated.

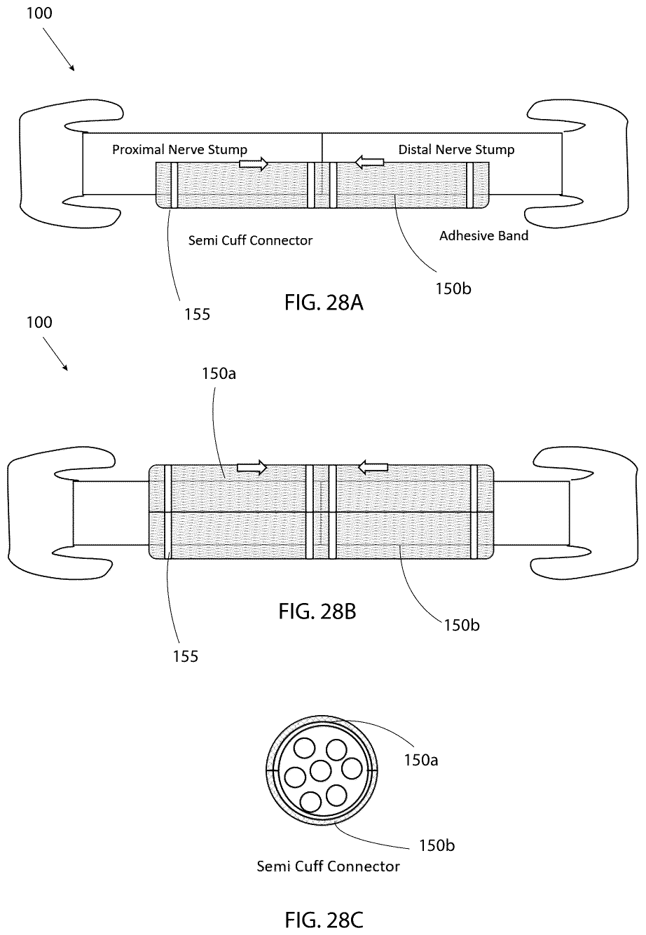

Referring now to FIGS. 28A-C, anatomical side views of an embodiment of a neurorrhaphy device is illustrated, consistent with the present inventive concepts. Device 100, as shown at least partially deployed at a nerve transection in FIGS. 28A,B, can comprise at least two fixation elements 150, such as 150a,b shown, each comprising a semi-cuff constructed and arranged to slidingly receive at least a portion of each nerve stump. As shown, a first portion of fixation elements 150a,b can slidingly receive the proximal nerve stump and a second portion of fixation elements 150a,b can slidingly receive the distal nerve stump. As shown in FIG. 28C, fixation elements 150a,b can be collectively constructed and arranged to enclose a circumference (e.g. full circumference) of the nerve stumps, thereby providing additional stability and support to the approximated nerves.

Fixation elements 150a,b can comprise one, two, or more anchoring elements 155 configured to attach fixation elements 150a,b to the nerve stumps. As shown, anchoring element 155 can comprise adhesive bands configured to adhere to tissue proximate the nerve stumps. Anchoring element 155 can comprise an adhesive selected from the group consisting of: biologically derived adhesive, such as fibrin glue, animal or fish-based adhesives; mucoadhesive; chemical adhesive, such as polyacrylic acid-based adhesives, or polyurethane-based adhesives; and combinations of these. Anchoring element 155 can comprise biologically derived materials configured to photochemically, thermally, and/or chemically bond fixation elements 150a,b to the nerve stumps.

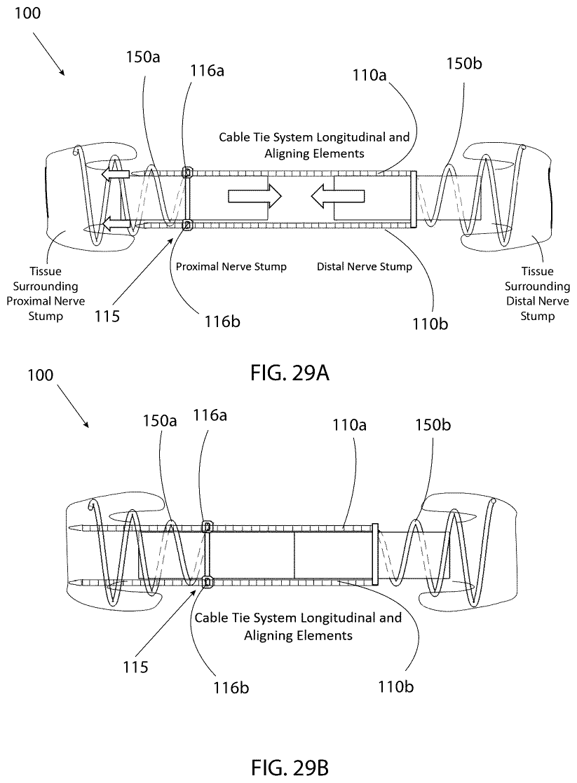

Referring now to FIGS. 29A and 29B, anatomical side views of an embodiment of a neurorrhaphy device is illustrated, consistent with the present inventive concepts. Device 100 can, shown at least partially deployed at a nerve transection site in FIGS. 29A,B, comprise two or more longitudinal elements 110, such as 110a,b shown, and two or more fixation elements 150, such as 150a,b shown. Device 100 can comprise tension adjusting element 115 coupled (e.g. mechanically coupled) between longitudinal elements 110a,b, as shown. Fixation element 150a can be applied to tissue proximate the proximal nerve stump and fixation element 150b can be applied to tissue proximate the distal nerve stump, with longitudinal elements 110a,b extending therebetween. Longitudinal elements 110a,b can be coupled (e.g. mechanically coupled) to fixation elements 150a,b to stabilize the nerve injury.

As shown, fixation elements 150a,b can each comprise a helical coil which can be positioned concentrically about the proximal and distal nerve stumps, respectively. Rotation of device 100 (e.g. via a fixation element 150) about its longitudinal axis can cause fixation elements 150a,b to progressively penetrate into tissue of the respective nerve stump, thereby increasing the anchoring depth of fixation elements 150a,b into the tissue. In some embodiments, oppositely-positioned helical couplers have opposite pitch orientation (e.g. left coupler: right-handed pitch; right coupler: left-handed pitch).

The cumulative and/or discrete length of longitudinal elements 110a,b can be adjusted to a size needed to approximate and/or align the proximal and distal nerve stumps. In some embodiments, the cumulative and/or discrete length of longitudinal elements 110a,b can be readjusted after implantation into the patient, such as when the initial length was improperly estimated prior to implantation. In some embodiments, the cumulative length of longitudinal elements 110a,b is adjustable via a cable tie system. The distal ends of longitudinal elements 110a,b can be coupled to the proximal end of fixation element 150b. Tension adjusting element 115 can be coupled to the proximal end of fixation element 150a. The proximal ends of longitudinal elements 110a,b can be thread through, and secured to, frames 116a,b of tension adjusting element 115. As longitudinal elements 110a,b are thread further through frames 116a,b, the distance between the proximal and distal nerves stumps is reduced, thereby allowing for the reapproximation of the nerve stumps.

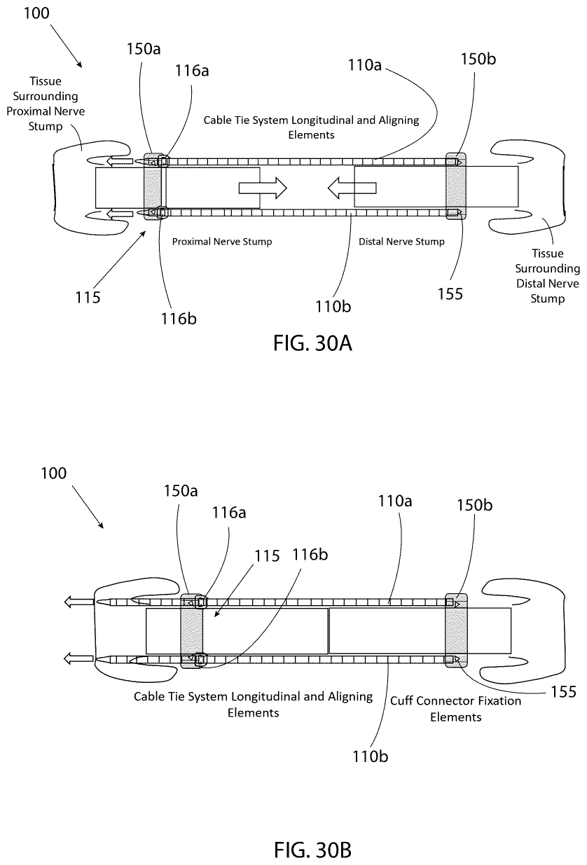

Referring now to FIGS. 30A and 30B, anatomical side views of an embodiment of a neurorrhaphy device is illustrated, consistent with the present inventive concepts. Device 100 can, shown at least partially deployed at a nerve transection site in FIGS. 30A,B, comprise two or more longitudinal elements 110, such as 110a,b shown, and two or more fixation elements 150, such as 150a,b shown. Device 100 can comprise tension adjusting element 115 coupled (e.g. mechanically coupled) between longitudinal elements 110a,b, as shown. Fixation element 150a can be applied to tissue proximate the proximal nerve stump and fixation element 150b can be applied to tissue proximate the distal nerve stump, with longitudinal elements 110a,b extending therebetween. Longitudinal elements 110a,b can be coupled (e.g. mechanically coupled) to fixation elements 150a,b to stabilize the nerve injury.

Fixation elements 150a,b can each comprise one, two, or more anchoring elements 155 configured to attach fixation element 150 to the nerve stump. As shown, anchoring element 155 can comprise barbs constructed and arranged to penetrate the nerve stump surface (e.g. nerve epineurium). Anchoring element 155 can comprise barbs configured to resist relative rotation between fixation element 150 and the respective nerve stump. In some embodiments, anchoring element 155 comprises bidirectional barbs, such that bidirectional longitudinal friction is created once the nerve stump is inserted into fixation element 150. In some embodiments, anchoring element 155 comprises unidirectional barbs, such that unidirectional longitudinal friction is created once the nerve stump is inserted into fixation element 150.

The cumulative and/or discrete length of longitudinal elements 110a,b can be adjusted to a size needed to approximate and/or align the proximal and distal nerve stumps. In some embodiments, the cumulative and/or discrete length of longitudinal elements 110a,b can be readjusted after implantation into the patient, such as when the initial length was improperly estimated prior to implantation. In some embodiments, the cumulative length of longitudinal elements 110a,b is adjustable via a cable tie system. The distal ends of longitudinal elements 110a,b can be coupled to the proximal end of fixation element 150b. Tension adjusting element 115 can be coupled to the proximal end of fixation element 150a. The proximal ends of longitudinal elements 110a,b can be thread through, and secured to, frames 116a,b of tension adjusting element 115. As longitudinal elements 110a,b are thread further through frames 116a,b, the distance between the proximal and distal nerves stumps is reduced, thereby allowing for the reapproximation of the nerve stumps.

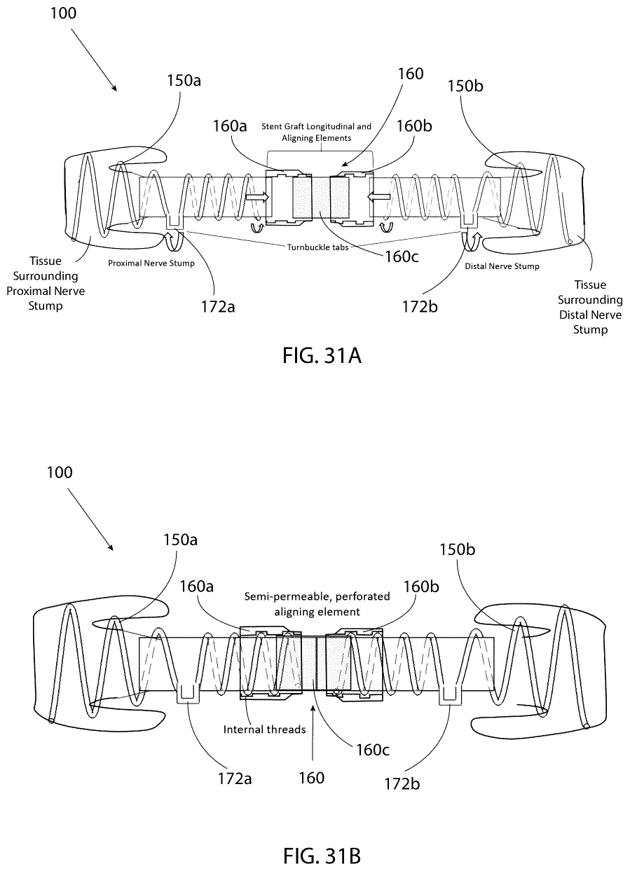

Referring now to FIGS. 31A and 31B, anatomical side views of an embodiment of a neurorrhaphy device is illustrated, consistent with the present inventive concepts. Device 100, shown at least partially deployed at a nerve transection site in FIGS. 31A,B, can comprise two or more fixation elements 150, such as 150a,b shown, and an aligning element 160. Fixation element 150a can be applied to the proximal nerve stump and fixation element 150b can be applied to the distal nerve stump, with aligning element 160 extending therebetween. Device 100 can further comprise one, two, or more coupling elements 172, such as 172a,b shown, configured to assist in a rotation of fixation elements 150a,b.

Fixation elements 150a,b can each comprise a helical coil which can be positioned concentrically about the proximal and distal nerve stumps, respectively.

Aligning element 160 can comprise a first portion 160a, a second portion 160b, and a middle portion 160c extending therebetween. First portion 160a can be configured to receive at least a portion of the proximal nerve stump and/or fixation element 150a, and second portion 160b can be configured to receive at least a portion of the distal nerve stump and/or fixation element 150b. Middle portion 160c can comprise one, two, or more semi-permeable, translucent, and/or perforated materials configured to facilitate regeneration between the proximal and distal nerve stumps. In some embodiments, the permeability of middle portion 160c (e.g. size and/or frequency of perforations within middle portion 160c) can be altered to accommodate one, two, or more characteristics of the nerve stump tissue.

Device 100 can comprise a pitch and/or inner diameter constructed and arranged to vary along its length. For example, the distal ends of fixation elements 150a,b can comprise a conical shape comprising a larger pitch, such that device 100 can be securely anchored to tissue surrounding the nerve stumps while the proximal ends of fixation elements 150a,b can comprise a smaller pitch around the region of coaptation such that the nerve stumps can be held tightly in their realigned position. In some embodiments, the pitch orientations along the length of device 100 are constructed and arranged to transition from a first pitch orientation (e.g. left-handed pitch) to a second pitch orientation (e.g. right-handed pitch) via a coupling element 172.

Rotation of fixation elements 150a,b (e.g. via at least one of coupling elements 172a,b) about their longitudinal axis can cause fixation elements 150a,b to progressively penetrate into tissue, thereby increasing the anchoring depth of fixation elements 150a,b into the tissue. In some embodiments, fixation elements 150a,b are constructed and arranged to comprise opposite pitch orientations (e.g. fixation element 150a comprises a left-handed pitch and fixation element 150b comprises a right-handed pitch, or vice versa).

Rotation of fixation elements 150a,b (e.g. via at least one of coupling elements 172a,b) about their longitudinal axis can cause the proximal ends of fixation elements 150a,b to rotationally and progressively engage aligning elements 160a,b, respectively. As fixation elements 150a,b are advanced further within aligning elements 160a,b, the distance between the proximal and distal nerve stumps decreases, thereby allowing for the reapproximation of the nerve stumps. In some embodiments, aligning element 160 is rotated to simultaneously engage the free ends of fixation elements 150a,b, decreasing the distance between the proximal and distal nerve stumps, thereby allowing for the reapproximation of the nerve stumps.

In some embodiments, fixation elements 150a,b comprise shape memory Nitinol. Fixation elements 150a,b can be progressively engaged within aligning element 160a,b, after which the shape memory function of fixation elements 150a,b is activated to thereby shorten device 100 to a length that is configured to fully reapproximate the proximal and distal nerve stumps.

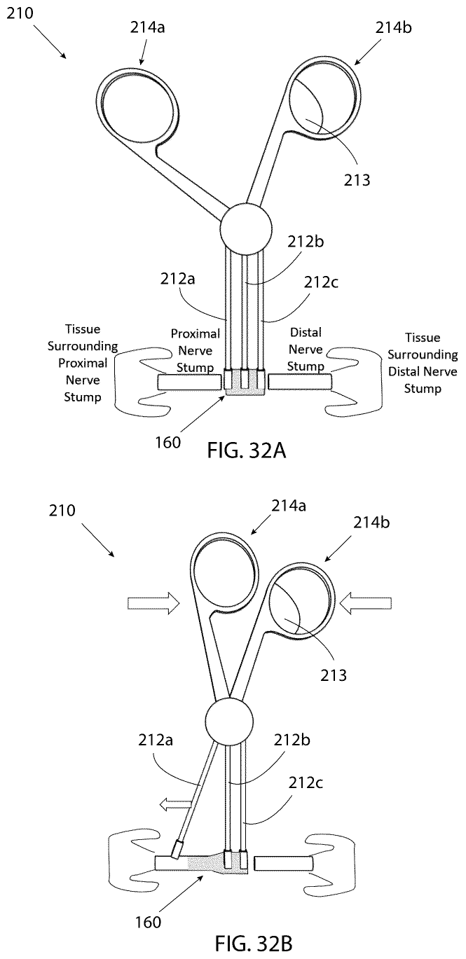

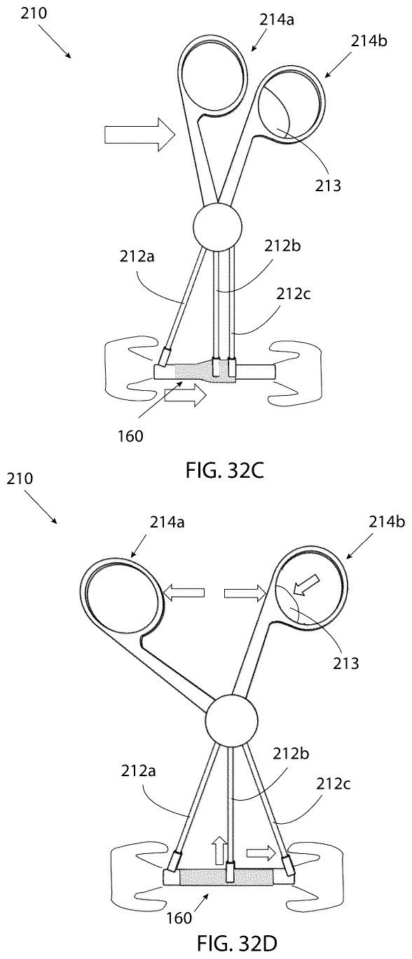

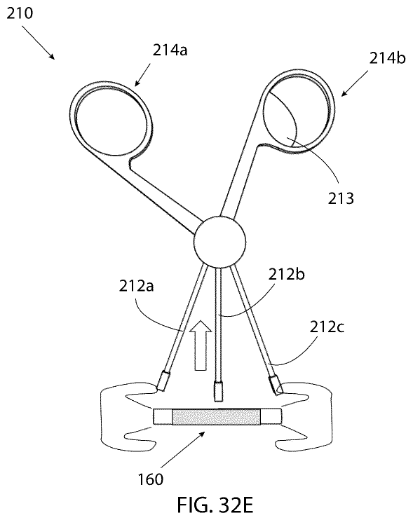

Referring now to FIGS. 32A-E, schematic views of an embodiment of a tool for holding and deploying a neurorrhaphy device 100 are illustrated, consistent with the present inventive concepts. Deployment tool 210 can be constructed to hold and deploy a device 100 at a nerve transection site. Deployment tool 210 as shown in FIGS. 32A-E can be constructed to hold and deploy device 100 in the embodiment as described herein in reference to FIG. 13A,B or 14A-D, wherein device 100 comprises an aligning element 160 comprising a braided cuff constructed and arranged to slidingly receive at least a portion of the proximal and distal nerve stumps.

Deployment tool 210 can be constructed and arranged to hold a preloaded aligning element 160 in a compressed state along its main axis and control the extension in length, concurrent reduction in diameter, and release of aligning element 160. As described hereinbelow, deployment tool 210 comprises at least three extendable arms 212, arms 212a-c shown, controlled via a set of handles 214a,b and a central static support arm 212c. In some embodiments, at least one handle 214 further includes a release trigger 213.

Referring specifically to FIG. 32A, a user (e.g. clinician) holds deployment tool 210 via handles 214a,b with a preloaded and compressed aligning element 160 proximate to the site of nerve transection. In some embodiments, deployment tool 210 further compresses one, two, or more fixation elements 150 (not shown) via arms 212a-c, thereby maintaining an increased inner diameter of the aligning element 160.

Referring specifically to FIG. 32B, the clinician concentrically aligns the proximal nerve stump with the proximal end of aligning element 160. The clinician extends the compressed, proximal end of aligning element 160 by squeezing handles 214a,b, allowing for the elongation in length and subsequent reduction in diameter of aligning element 160. The proximal portion of aligning element 160 is released from deployment tool 210 and engages the proximal nerve stump of the transected nerve when handles 214a,b are parted (e.g. minimally separated).

Referring specifically to FIG. 32C, once aligning element 160 engages the proximal nerve stump, deployment tool 210 is positioned to approximate, align, and coapt the proximal and distal nerve stumps.

Referring specifically to FIG. 32D, once the proximal and distal nerve stumps are desirably approximated, aligned, and coapted, trigger 213 on handle 214b is depressed by the clinician, allowing handles 214a,b to be separated again. The separation of handles 214a,b allows the clinician to extend the remaining compressed, distal end of aligning element 160 over the distal stump, allowing for the elongation in length and subsequent reduction in diameter of aligning element 160. The distal and central portions of aligning element 160 are released from deployment tool 210 and engage the distal nerve stump of the transected nerve, thereby securing the coaptation of the proximal and distal nerve stumps.

Referring specifically to FIG. 32E, the clinician removes deployment tool 210 from the nerve transection site.

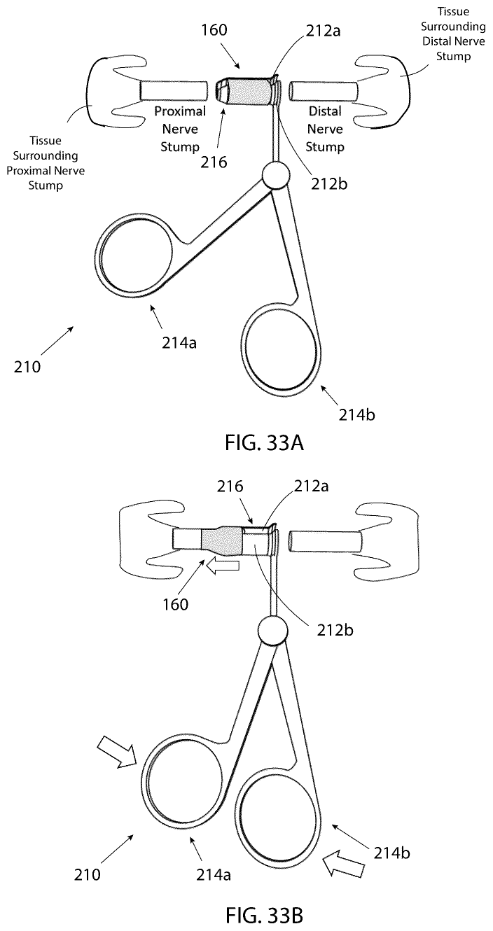

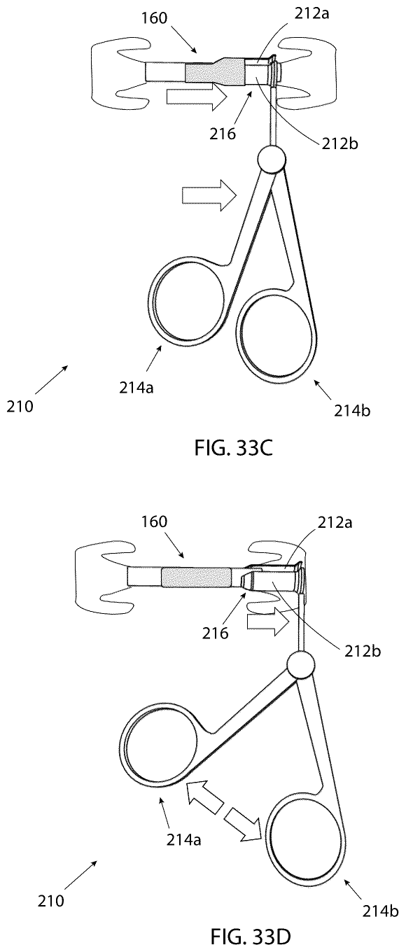

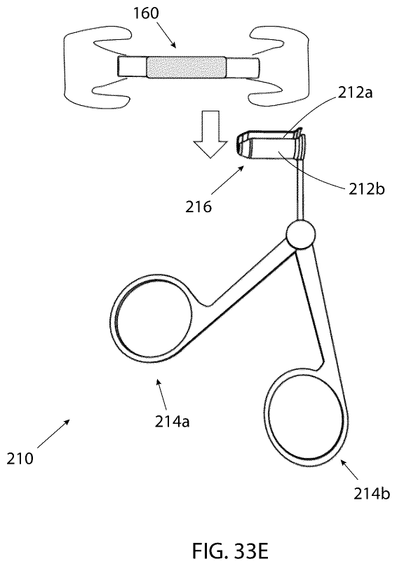

Referring now to FIGS. 33A-E, schematic views of an embodiment of a tool for holding and deploying a neurorrhaphy device 100 are illustrated, consistent with the present inventive concepts. Deployment tool 210 can be constructed and arranged to hold and deploy a device 100 at a nerve transection site. Deployment tool 210 as shown in FIGS. 33A-E can be constructed and arranged to hold and deploy device 100 in the embodiments as described herein in reference to FIGS. 13A,B or FIGS. 14A-D, wherein device 100 comprises aligning element 160 comprising a braided cuff constructed and arranged to slidingly receive at least a portion of the proximal and distal nerve stumps.

Deployment tool 210 can be constructed and arranged to hold a preloaded aligning element 160 in a compressed state along its main axis and control the extension in length, concurrent reduction in diameter, and release of aligning element 160. As described hereinbelow, deployment tool 210 comprises a housing 216 and at least two extendable arms 212, arms 212a,b shown, controlled via a set of handles 214a,b for the control and positioning of deployment tool 210 at the nerve transection site. Housing 216 is configured to be slidingly received via a lumen of aligning element 160, such that aligning element 160 is preloaded onto deployment tool 210. In some embodiments, housing 216 comprises a hollow cylinder comprising at least one tapered end. In some embodiments, deployment tool 210 further comprises a release mechanism configured to release aligning element 160 from housing 216.

Referring specifically to FIG. 33A, a user (e.g. clinician) holds deployment tool 210 with preloaded aligning element 160 via handles 214a,b proximate to the site of nerve transection.

Referring specifically to FIG. 33B, the clinician concentrically aligns the proximal nerve stump with the tapered end of housing 216. The clinician squeezes handles 214a,b to progressively extend and release a first portion of aligning element 160 over the proximal nerve stump, while retaining the remaining portion of aligning element 160 on housing 216.

Referring specifically to FIG. 33C, the clinician uses deployment tool 210, engaged at the proximal nerve stump by the aligning element 160, to pull, align, and coapt the proximal nerve stump with the distal nerve stump.

Referring specifically to FIG. 33D, the clinician separates handles 214a,b while retracting the deployment tool 210 over the distal nerve stump to progressively extend and release the remaining portion of aligning element 160 over the distal nerve stump until its fully released from housing 216, thereby allowing permanent engagement between the two nerve stumps.

Referring specifically to FIG. 33E, deployment tool 210 is the removed from the nerve transection site.

Device 100 can be constructed and arranged to exhibit a desired “Dynamic Range” of properties tailored to specific nerve sizes, applications, usability, durability, effectiveness, and/or other requirements. Dynamic Range describes the correlation between the radial and longitudinal deformations of device 100, and its resulting dynamic responses. For example, Dynamic Range describes the relationship between changes in the length of device 100 and the resulting changes in diameter and forces exhibited by the device. Thus, Dynamic Range constitutes a fundamental intrinsic characterization of device 100, which allows for the tailored design and the in vitrolex vivo/in vivo properties verification of device 100 for its intended use. As described herein in reference to FIGS. 14A-D, Dynamic Range can be used to characterize an aligning element 160 comprising a braided cuff constructed and arranged to slidingly receive at least a portion of the proximal and distal nerve stumps for its intrinsic geometrical and mechanical properties. Dynamic Range describes the concurrent increase in length and reduction in internal diameter of aligning element 160 and the associated tensile forces resulting from such elongation from its neutral state (e.g. when no external forces are applied to aligning element 160; “Tensile Dynamic Range” herein). Dynamic Range also describes the concurrent decrease in length and increase in internal diameter of aligning element 160 and the associated compressive forces resulting from such compression from its neutral state (e.g. when no external forces are applied to aligning element 160; “Compressive Dynamic Range” herein).

In some embodiments, aligning element 160 can be designed and constructed to exhibit a specific Tensile Dynamic Range in which limited extension in length from its neutral state results in a decrease in diameter and an increase in tensile forces.

In some embodiments, aligning element 160 can be designed and constructed to exhibit a specific Compressive Dynamic Range in which aligning element 160 is compressed along its central axis, resulting in an increase (e.g. significant increase) in internal diameter and a decrease (e.g. significant decrease) in length from its neutral state. Compressive Dynamic Range can facilitate the deployment of aligning element 160 as it is slidingly receives the nerve stumps.

Desired Tensile and/or Compressive Dynamic Ranges can be achieved by constructing aligning element 160 with specific braiding parameters. For example, a low braid angle (e.g. an angle of between 5 and 50 degrees) can be configured to provide aligning element 160 with a wide Compressive Dynamic Range and/or a narrow Tensile Dynamic Range. Manipulation of the Tensile and/or Compressive Dynamic Ranges allows for aligning element 160 to slidingly receive the nerve stumps and provide significant tensile support to maintain nerve coaptation.

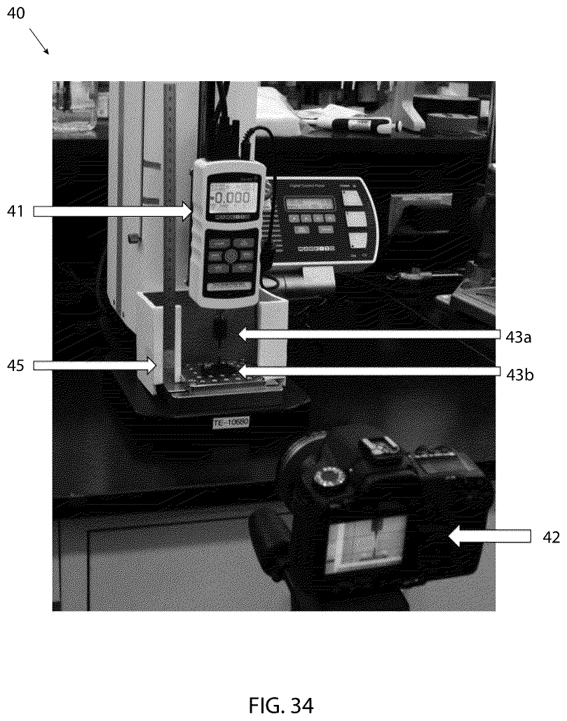

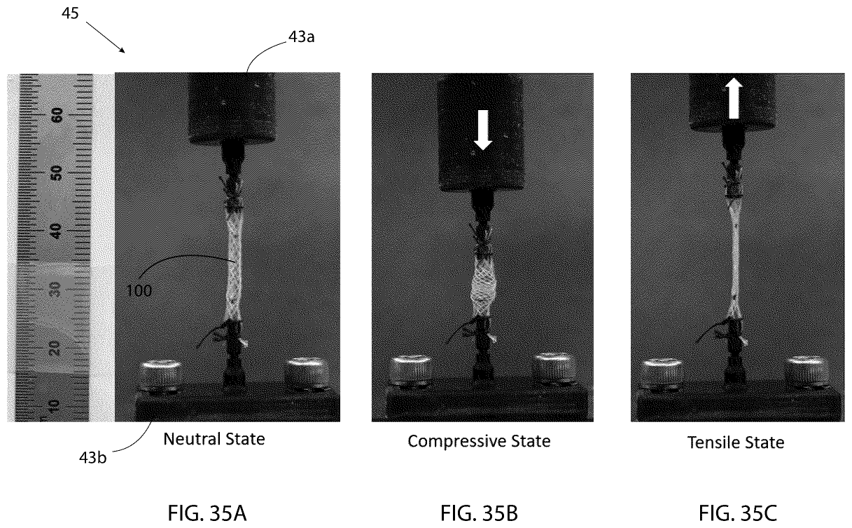

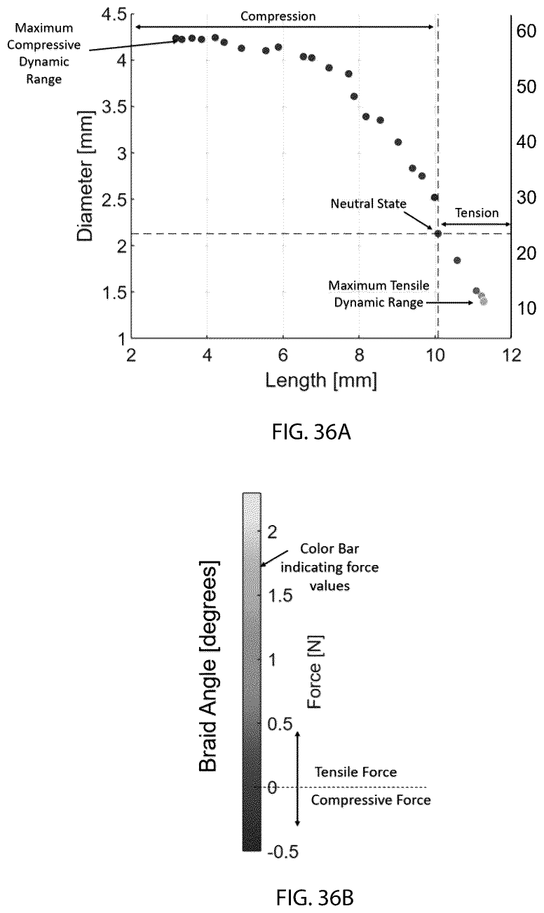

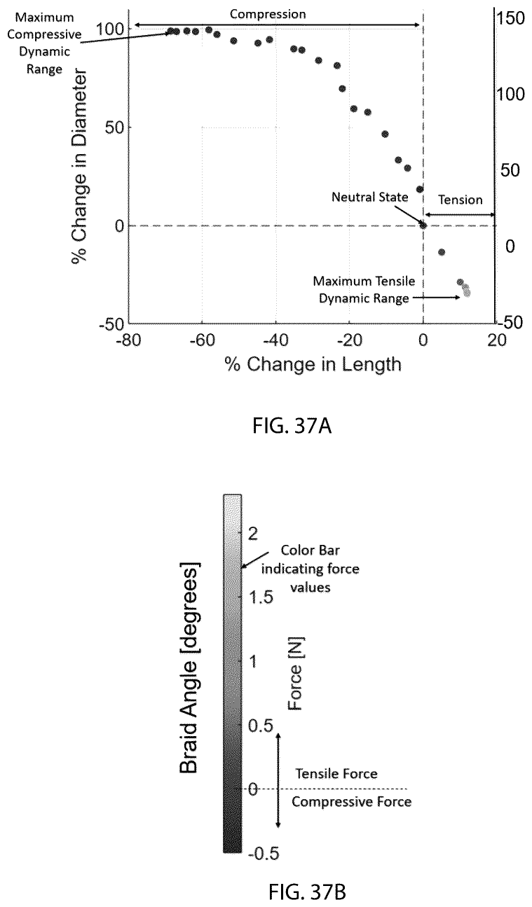

Referring now to FIG. 34 thru FIGS. 37A and 37B, an embodiment of a system for testing and/or verifying functional characteristics of a neurorrhaphy device in vitro and example output data are illustrated, consistent with the present inventive concepts. The Dynamic Range Testing System, testing system 40 herein, can be constructed and arranged to test and/or verify the Tensile and/or Compressive Dynamic Ranges of device 100 in vitro. As described hereinbelow, device 100 comprises aligning element 160 comprising a braided cuff constructed and arranged to slidingly receive at least a portion of the proximal and distal nerve stumps, and as described herein in reference to FIGS. 13A,B and/or FIGS. 14A-D.

System 40 can include a uniaxial testing system apparatus 41, a visual recording device 42, a device support 43, and/or a nerve support 44. System 40 can further include a vertical field of view background 45 comprising a length scale positioned planarly with the central axis of aligning element 160 to be tested.

Apparatus 41 can comprise a programmable motorized linear stage controlled by a digital controller, actuated by servo or stepper motors with sensors collecting real-time position/displacement data, and a digital force gauge mounted to the motorized linear stage collecting real-time force data.

Device 42 can comprise a high-definition video camera with a macro photography lens configured to acquire videos in focus for large depths of field. Device 42 can be positioned orthogonally to the central axis of apparatus 41 and to background 45 to image the testing field throughout the duration of Dynamic Range testing. In some embodiments, device 42 is configured to track the positions of one, two, or more reference points (e.g. tracking beads) distributed over the surface of aligning element 160 being tested. Device 42 can be configured to measure changes in local or generalized strains in the vertical and/or horizontal directions of aligning element 160 being tested, enabling device 42 to record changes in the length and diameter of aligning element 160 over time. Device 42 can be configured to enable the dynamic measurement of localized strains, changes in fiber alignments, and/or full reconstruction of three-dimensional geometry of aligning element 160 over the duration of Dynamic Range testing.

Device support 43 can comprise one, two, or more textured cylindrical posts comprising a diameter similar to the inner diameter of the aligning element 160 to be tested. Device support 43 can be mounted along the central axis of apparatus 41. In some embodiments, a first device support 43a is mounted to a force gauge of apparatus 41 and a second device support 43b is mounted to a base plate of apparatus 41, as shown in FIG. 34.

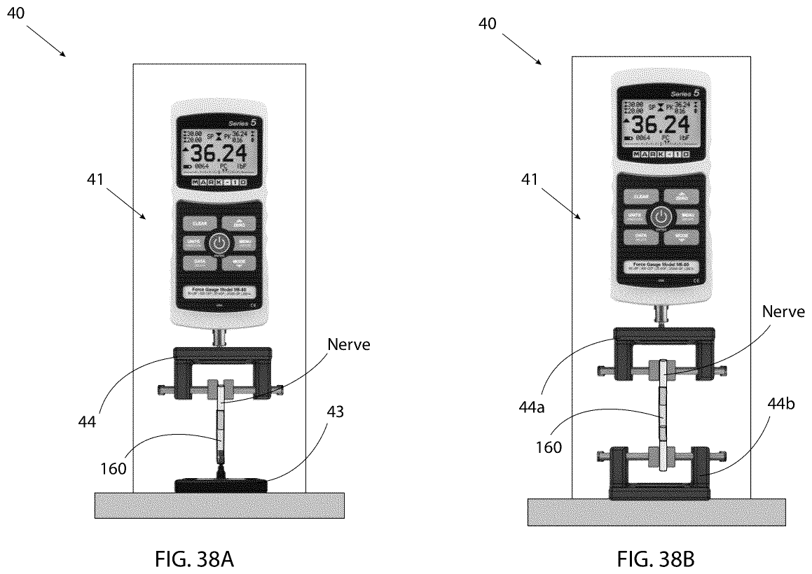

Nerve support 44 can comprise one, two, or more textured, adjustable vice or chuck grips mounted along the central axis of apparatus 41. In some embodiments, a first nerve support 44a is mounted to a force gauge of apparatus 41 and a second nerve support 44b is mounted to a base plate of apparatus 41, as shown in FIG. 38B.

In some embodiments, system 40 further includes a servo or stepper motor 46 (not shown) and a separate torque transducer 47 (not shown). Motor 46 and transducer 47 can be configured to collectively apply a controlled rotational motion around the main axis of aligning element 160 at a first end and record the resulting torsional forces.

In some embodiments, system 40 further includes a transparent, water-tight tank 48 (not shown) to hold a fluid (e.g. saline) within the central axis of apparatus 41. System 40 can further include a temperature control system 49 (not shown) to maintain the fluid within tank 48 at a defined temperature, such as a temperature of approximately 37° C.

Referring specifically to FIGS. 34, an exemplary system 40 comprises apparatus 41, device 42, and device supports 43a,b. Additionally, background 45 comprising a length scale is positioned planarly with the central axis of apparatus 41.

Referring specifically to FIG. 35A, Dynamic Range testing of aligning element 160 can be performed by mounting aligning element 160 in a neutral state to device supports 43a,b and secured with suture ligations.

Referring specifically to FIG. 35B, from the neutral state as shown in FIG. 35A, a crosshead of apparatus 41 is configured to descend at a speed of between 10 and 200 mm/min, thereby axially compressing aligning element 160 until device 42 records a plateau in the diameter of aligning element 160.

Referring specifically to FIG. 35C, from the compressive state as shown in FIG. 35B, the crosshead of apparatus 41 ceases movement and ascends at a speed of between 10 and 200 mm/min until the force transducer records a significant gradient in force. This sudden increase in force suggests alignment element 160 has reached its full longitudinal extension and corresponding minimum diameter. This state is achieved when the braided threads of aligning element 160 have reached a critical vertical alignment with a minimum pitch angle. In some embodiments, multiple serial cycles of Dynamic Range Testing can be performed on the same aligning element 160. For example, multiple cycles of Dynamic Range Testing can be configured to precondition (e.g. relieve internal residual stresses) of aligning element 160. In some embodiments, Dynamic Range Testing is performed under physiologic conditions in saline at 37° C. for extensive durations and/or number of cycles to assess the durability of aligning element 160 (e.g. fatigue testing). In some embodiments, the system 40 is configured to test aligning element 160 beyond its compressive and tensile limits to assess its ultimate compressive and tensile properties.

Referring specifically FIGS. 36A and 36B, an example of data collected from Dynamic Range testing of an aligning element 160 constructed by braiding 16 5-0 plain gut suture threads in a 2:2 pattern with a neutral outer diameter 1.85 mm is shown. In particular, the length, diameter, braid angle, and force associated with aligning element 160 are shown over the duration of the Dynamic Range Testing.

Referring specifically to FIGS. 37A and 37B, an example of data collected from Dynamic Range testing of an aligning element 160 constructed by braiding 16 5-0 plain gut suture threads in a 2:2 pattern with a neutral outer diameter 1.85 mm is shown. In particular, the length, diameter, and braid angle from the Dynamic Range Testing of aligning element 160 described herein in reference to FIGS. 36A,B, are shown as percent changes from the neutral state of aligning element 160.

Referring now to FIGS. 38A and 38B, an embodiment of a system for testing and/or verifying functional characteristics of a neurorrhaphy device ex vivo are illustrated, consistent with the present inventive concepts. The performance of device 100 can be characterized via ex vivo testing of a partially deployed device 100 onto a peripheral porcine nerve segment. As described herein, device 100 comprises aligning element 160 comprising a braided cuff constructed and arranged to slidingly receive at least a portion of the proximal and distal nerve stumps, and as described herein in reference to FIGS. 13A,B and/or FIGS. 14A-D.

A modified Dynamic Range Testing System, modified system 40 herein, can be constructed and arranged similar to system 40 described hereinabove in reference to FIG. 34 thru FIGS. 37A,B. Modified system 40 can be constructed and arranged to assess the ex vivo performance of an aligning element 160 by replacing one or both device supports 43a,b with one or both nerve supports 44a,b. Modified system 40 can be configured to assess the ability of aligning element 160 to support the longitudinal force to maintain the nerve stump coaptation.

In some embodiments, modified system 40 further includes motor 46 and transducer 47. Dynamic Range Testing can be configured to assess the ability of aligning element 160 torsional forces to maintain the nerve stump coaptation.

In some embodiments, modified system 40 is mounted with the transparent, water-tight tank 48 to hold a fluid (e.g. saline) within the central axis of uniaxial testing system apparatus 41. Temperature control system 49 maintains the fluid within tank 48 at a defined temperature of approximately 37° C. to measure the ability of aligning element 160 to support the longitudinal and torsional forces necessary to maintain nerve stump coaptation under physiological conditions.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the inventive concepts, which is defined in the accompanying claims.

1.-107. (canceled)

108. A system for connecting two nerve stumps, comprising:
a device, the device comprising:
at least one longitudinal element, at least two fixation elements and at least one aligning element;
wherein the system is configured to be deployed in at least one deposit site within a patient and wherein the longitudinal element, the fixations elements, and the aligning element cooperate to connect a proximal nerve stump and a distal nerve stump without the use of sutures and glues at and proximate to a nerve coaptation site; thereby improving the ease, reproducibility, and/or speed of connecting the proximal nerve stump and the distal nerve stump.

109. The system according to claim 108, wherein the device is configured to maintain a calculated gap length between the proximal nerve stump and the distal nerve stump to promote or to facilitate nerve growth and/or axonal alignment between the proximal nerve stump and the distal nerve stump.

110. The system according to claim 108, wherein the device is comprised of materials that can be configured to degrade without eliciting significant inflammatory and/or foreign body reaction.

111. The system according to claim 108, wherein the device comprises a durable and/or degradable spacer between nerve stumps, which can comprise biological and/or synthetic material configured to promote and/or to facilitate nerve growth and/or axonal alignment between the proximal nerve stump and the distal nerve stump.

112. The system according to claim 108, wherein the at least one longitudinal element comprises at least one tension adjusting element configured to adjust tension and distance between the proximal nerve stump and the distal nerve stump.

113. The system according to claim 108, wherein the at least one longitudinal element, and/or the at least two fixation elements, and/or at least one aligning element comprises one or more durable metals, alloys, and/or polymers, and/or biological materials, and combination thereof.

114. The system according to claim 108, wherein the at least one longitudinal element, and/or the at least two fixation elements, and/or at least one aligning element comprises one, two, or more elements selected from the group consisting of: cylindrical coil; cylindrical clam;
cylindrical mesh/cuff obtained by braiding, knitting, or weaving; longitudinally and/or partially or completely circumferentially-interlocking elements; a spacer between the proximal nerve stump and the distal nerve stump; and combinations thereof.

115. The system according to claim 108, wherein the at least two fixation elements are each comprised of an anchoring element, the anchoring element can comprise an element selected from the group consisting of: plow tether; coil, hook; clip; buttonhole; cringle; grommet; barb; adhesive; and combinations thereof.

116. The system according to claim 108, wherein the at least two fixation elements are constructed to be directly tethered to the proximal nerve stump and the distal nerve stump.

117. The system according to claim 108, wherein the at least two fixation elements are constructed and arranged to create one or more anchoring points onto a nerve surface, within tissue adjacent to, proximate to, or surrounding the nerve stumps, or in combinations thereof.

118. The system according to claim 108, wherein the at least two fixation elements can comprise one more biologically derived material, such as human amniotic membrane, blood vessels, umbilical cord vessels, and nerve tissue.

119. The system according to claim 108, wherein the at least two fixation elements and/or the at least one aligning element comprises at least one surface with a texture configured to improve its frictional properties to maintain stability, while other surfaces are treated to reduce frictional properties.

120. The system according to claim 108, wherein the device is further configured to provide visibility of the deposit site, such as to ensure correct longitudinal distancing and/or circumferential alignment between the proximal nerve stump and the distal nerve stump.

121. A system for connecting two nerve stumps, comprising:
a braided mesh, the braided mesh being comprised of a lumen with an internal diameter, a length, a first end opening, and a second end opening, the braided mesh being capable of receiving and surrounding a portion of a proximal nerve stump at the first end opening within the lumen and being capable of receiving and surrounding a portion of a distal nerve stump at the second end opening within the lumen,
wherein the braided mesh maintains approximation and aligns the proximal nerve stump and the distal nerve sump without the use of sutures and glues at and proximate to a nerve coaptation site, thereby improving the ease, reproducibility, and/or speed of connecting the proximal nerve stump and the distal nerve stump;
wherein the braided mesh is configured to controllably alter its internal diameter of the lumen when longitudinal/axial tension and stretch are applied to the braided mesh allowing the braided mesh to be capable of resisting longitudinal and torsional forces keeping the proximal nerve stump and the distal nerve stump at a defined distance and alignment.

122. The system of claim 121, wherein following axial compression of the braided mesh upon the first end opening receiving the proximal nerve stump and upon the second end opening receiving the distal nerve stump, a release of said axial compression with rotational alignment while holding the proximal nerve stump and the distal nerve stump at a desired distance allows the braided mesh to return to a neutral state in which the length of the braided mesh is sufficient to overlap portions of the proximal nerve stump and the distal nerve stump; and
whereby the internal diameter is smaller than that of either the proximal nerve stump or the distal nerve stump.

123. The system according to claim 121, wherein the braided mesh is constructed from suture strands of size between 2-0 and 6-0 degradable and/or non-degradable sutures by braiding 8, 16, 24, 32, or 48 suture threads in a 1:1, 1:2, or 2:2 relation, with a braid angle of between 5 and 85 degrees, and wherein the braid angle defines a braid pick per unit length, wherein the mesh comprises a porosity of between 20% and 60%.

124. The system according to claim 121, wherein the braided mesh is constructed from a plurality of monofilament threads obtained from extracellular matrices.

125. The system according to claim 121, wherein the device is configured to maintain a calculated gap length between the proximal and distal nerve stumps to promote or to facilitate nerve growth and/or axonal alignment between the proximal nerve stump and the distal nerve stump.

126. The system according to claim 121, wherein the braided mesh comprises at least one surface treated with a texture configured to improve frictional properties to maintain stability, while other surfaces are treated to reduce frictional properties.

127. The system according to claim 121, where the braided mesh further comprises anchoring elements.

128. The system for connecting two nerve stumps, comprising a device, the device comprising a braided mesh and at least two fixation elements; the braided mesh being comprised of a first end and a second end; the at least two fixation elements being comprised of a first fixation element and a second fixation element,
the braided mesh, the braided mesh being comprised of a lumen with an internal diameter, a length, a first end opening, and a second end opening, the braided mesh being capable of receiving and surrounding a portion of a proximal nerve stump at the first end opening within the lumen and being capable of receiving and surrounding a portion of a distal nerve stump at the second end opening within the lumen;
wherein the first fixation element is comprised of a right-handed pitch helical coil coupled to the mesh at the first end, and wherein the second fixation element is comprised of a left-handed pitch helical coil coupled to the braided mesh at the second end, each of the right-handed pitch helical coil and the left-handed pitch helical having a lumen;
such that upon inserting each of the proximal nerve stump and the distal nerve stump within the respective lumen of each of the helical coils and within the lumen of the braided mesh, rotating the device in one direction produces a progressive and controlled approximation and alignment of the nerve stumps, whereas rotating the device in the opposite direction produces an increase distance between the two nerve stumps and reduces/eliminates the stump alignment until the release of each fixation device from the tissue.
